(19) United States
(12) Patent Application Publication
(10) Pub. No.: US 2017/0119295 A1
(43) Pub. Date: May 4, 2017

(54) Automated Scientifically Controlled Screening Systems (ASCSS)

(71) Applicant: The Arizonia Board of Regents on Behalf of the University of Arizonia, Tucson, AZ (US)

(72) Inventors: Nathan W. Twyman, Rolla, MO (US); Jay F. Nunamaker, Tucson, AZ (US)

(21) Appl. No.: 15/306,083

(22) PCT Filed: May 27, 2015
(86) PCT No.: PCT/US15/32640

§ 371 Date: Oct. 21, 2016

Related U.S. Application Data
(60) Provisional application No. 62/003,541, filed on May 27, 2014.

(51) Int. Cl. A61B 5/16; A61B 3/11; A61B 5/00; A61B 3/113

(52) U.S. Cl. CPC A61B 5/164; A61B 3/113; A61B 3/112; A61B 5/4884

(57) ABSTRACT

The ability to detect when a person is hiding important information has high value in many applications. A new class of systems, termed autonomous scientifically controlled screening systems (ASCSS), is designed to detect individuals' purposely hidden information about target topics of interest. ASCSS represents a systematic synthesis of structured interviewing, orienting theory, defensive response theory, non-invasive psychophysiological measurement, and behavioral measurement. To evaluate and enhance the design principles, an automated screening kiosk (ASK) system was constructed. The ASK system has been used in a physical security screening scenario in which participants constructed and attempted to smuggle a fake improvised explosive device (IED). The ASK system results indicate that ASCSS enables more widespread application of credibility assessment screening systems.

### CROSS-REFERENCE TO RELATED APPLICATIONS

The present application claims priority to U.S. Provisional Patent Application No. 62/003,541 entitled “Automated Scientifically Controlled Screening Systems (ASCSS)”, filed May 27, 2014, which is entirely incorporated by reference herein for all purposes.

### BACKGROUND OF THE INVENTION

Unless otherwise indicated herein, the materials described in this section are not prior art to the claims in this application and are not admitted to be prior art by inclusion in this section.

There are many circumstances when the intent and credibility of a person is rapidly and accurately determined. For example, transportation and border security systems have a common goal: to allow law-abiding people to pass through checkpoints and detain those people with hostile intent. These systems employ a number of security measures that are aimed at accomplishing this goal.

One example is when a person seeks entry into a country. At the border, the person can be interviewed to determine if they are importing goods into the country that require tax payment and/or may lead to harm to the country; e.g., explosives, guns, certain food products, soil from farms carrying microorganisms unknown to the country. The person can be interviewed about their intent upon entry to the country; e.g., questions about business or tourism plans, locations and persons within the country to be visited, etc.

In these cases, the questioning agents have to assess the credibility of the person seeking entry to decide if the person should be admitted to enter the country, or if some or all of their goods should be quarantined or taxed. However, having to make these assessments in a short time can fatigue even experienced agents. Further, even the most experienced agents can make incorrect assessments, which can lead to disgruntled entrants at best, and to possible security breaches at worst. For example, the general population of persons can detect lies at about a 54% success rate. Further, people often believe they are better lie detectors than these results warrant. Additionally, there may be significantly more persons seeking entrance to some locations of a country than there are agents available, leading to long delays in entry processing.

Achieving high information assurance is complicated not only by the speed, complexity, volume, and global reach of communications and information exchange that current information technologies now afford but by the fallibility of humans to detect non-credible persons with hostile intent. The agents guarding borders, transportation systems, and public spaces can be handicapped by untimely and incomplete information, overwhelming flows of people and materiel, and the limits of human vigilance.

The interactions and complex interdependencies of information systems and social systems render the problem difficult and challenging. Currently, there are not enough resources to specifically identify every potentially dangerous individual around the world. Although completely automating concealment detection is an appealing prospect, the complexity of detecting and countering hostile intentions defies a fully automated solution.

### SUMMARY

In one aspect, a method is provided. A computing device presents a plurality of stimuli to an observer. At least one stimulus of the plurality of stimuli is associated with particular information. The computing device uses a sensor associated with the computing device to obtain at least one measurement of the observer responding to the plurality of stimuli. The computing device makes a determination whether the observer had an orienting response and/or a defensive response to the plurality of stimuli based on the at least one measurement. The computing device determines a likelihood that the observer is aware of the particular information based on the determination whether the observer had the orienting response and/or the defensive response. The computing device provides an output based on the likelihood that the observer is aware of the particular information.

In particular aspects, the plurality of stimuli include visual stimuli. In more particular aspects, the plurality of stimuli include a plurality of images. In other particular aspects, the at least one measurement includes a measurement of eye movement of the observer. In still other particular aspects, making the determination whether the observer had the orienting response and/or the defensive response to the plurality of stimuli includes determining whether the observer had the orienting response based on an initial response to the plurality of stimuli. In particular of the still other particular aspects, the plurality of stimuli include a plurality of images, where a single particular image of the plurality of images is associated with the particular information, and where determining whether the observer had the orienting response based on an initial response to the plurality of stimuli includes determining whether an initial saccade of the observer is directed to the single particular image. In even other particular aspects, determining whether the observer had the orienting response and/or the defensive response to the plurality of stimuli includes: determining whether the observer had the defensive response based on a response to the plurality of stimuli. In particular of the even other particular aspects, the plurality of stimuli include a plurality of images, where a single particular image of the plurality of images is associated with the particular information, and where determining whether the observer had the defensive response includes determining whether the observer made at least one eye movement to a location away from the single particular image. In more particular of the even other particular aspects, presenting the plurality of stimuli includes presenting the plurality of images in locations with respect to a location of a fixation marker, where the location away from the single particular image includes the location of the fixation marker. In yet other particular aspects, providing the output of the computing device based on the likelihood that the observer is aware of the particular information includes: determining whether the likelihood that the observer is aware of the particular information is above a threshold likelihood; and after determining that likelihood that the observer is aware of the particular information is above the threshold likelihood, providing an output of the computing device recommending further screening of the observer. In additional particular aspects, providing the output of the computing device based on the likelihood that the observer is aware of the particular information includes: determining that the likelihood that the observer is aware of the particular information is not above a threshold likelihood; and after determining that the likelihood that the observer is aware of the particular information is not above the threshold likelihood, providing an output of the computing device indicating the observer likely does not have the particular information and so recommending no additional screening of the observer.

In another aspect, a computing device is provided. The computing device includes a sensor, a processor; and a tangible computer-readable medium. The tangible computer-readable medium is configured to store instructions that, when executed by the processor, are configured to cause the computing device to perform functions of a method. The functions include: presenting a plurality of stimuli to an observer, where at least one stimulus of the plurality of stimuli is associated with particular information; obtaining at least one measurement of the observer responding to the plurality of stimuli using the sensor; making a determination whether the observer had an orienting response and/or a defensive response to the plurality of stimuli based on the at least one measurement; determining a likelihood that the observer is aware of the particular information based on the determination whether the observer had the orienting response and/or the defensive response; and providing an output of based on the likelihood that the observer is aware of the particular information.

In particular aspects of the functions of the method, the plurality of stimuli include visual stimuli. In more particular aspects of the functions of the method, the plurality of stimuli include a plurality of images. In other particular aspects of the functions of the method, the at least one measurement includes a measurement of eye movement of the observer. In still other particular aspects of the functions of the method, making the determination whether the observer had the orienting response and/or the defensive response to the plurality of stimuli includes determining whether the observer had the orienting response based on an initial response to the plurality of stimuli. In particular of the still other particular aspects of the functions of the method, the plurality of stimuli include a plurality of images, where a single particular image of the plurality of images is associated with the particular information, and where determining whether the observer had the orienting response based on an initial response to the plurality of stimuli includes determining whether an initial saccade of the observer is directed to the single particular image. In even other particular aspects of the functions of the method, determining whether the observer had the orienting response and/or the defensive response to the plurality of stimuli includes: determining whether the observer had the defensive response based on a response to the plurality of stimuli. In particular of the even other particular aspects of the functions of the method, the plurality of stimuli include a plurality of images, where a single particular image of the plurality of images is associated with the particular information, and where determining whether the observer had the defensive response includes determining whether the observer made at least one eye movement to a location away from the single particular image. In more particular of the even other particular aspects of the functions of the method, presenting the plurality of stimuli includes presenting the plurality of images in locations with respect to a location of a fixation marker, where the location away from the single particular image includes the location of the fixation marker. In yet other particular aspects of the functions of the method, providing the output of the computing device based on the likelihood that the observer is aware of the particular information includes: determining whether the likelihood that the observer is aware of the particular information is above a threshold likelihood; and after determining that likelihood that the observer is aware of the particular information is above the threshold likelihood, providing an output of the computing device recommending further screening of the observer. In additional particular aspects of the functions of the method, providing the output of the computing device based on the likelihood that the observer is aware of the particular information includes: determining that the likelihood that the observer is aware of the particular information is not above a threshold likelihood; and after determining that the likelihood that the observer is aware of the particular information is not above the threshold likelihood, providing an output of the computing device indicating the observer likely does not have the particular information and so recommending no additional screening of the observer.

In particular aspects of the computing device, the tangible computer-readable medium includes a non-transitory computer-readable medium. In other particular aspects of the computing device, the computing device is configured to be operated in a kiosk.

In another aspect, a tangible computer-readable medium is provided. The tangible computer-readable medium is configured to store instructions that, when executed by a processor of a computing device, are configured to cause the computing device to perform functions of a method. The functions include: presenting a plurality of stimuli to an observer, where at least one stimulus of the plurality of stimuli is associated with particular information; obtaining at least one measurement of the observer responding to the plurality of stimuli using the sensor; making a determination whether the observer had an orienting response and/or a defensive response to the plurality of stimuli based on the at least one measurement; determining a likelihood that the observer is aware of the particular information based on the determination whether the observer had the orienting response and/or the defensive response; and providing an output of based on the likelihood that the observer is aware of the particular information.

In particular aspects of the functions of the method, the plurality of stimuli include visual stimuli. In more particular aspects of the functions of the method, the plurality of stimuli include a plurality of images. In other particular aspects of the functions of the method, the at least one measurement includes a measurement of eye movement of the observer. In still other particular aspects of the functions of the method, making the determination whether the observer had the orienting response and/or the defensive response to the plurality of stimuli includes determining whether the observer had the orienting response based on an initial response to the plurality of stimuli. In particular of the still other particular aspects of the functions of the method, the plurality of stimuli include a plurality of images, where a single particular image of the plurality of images is associated with the particular information, and where determining whether the observer had the orienting response based on an initial response to the plurality of stimuli includes determining whether an initial saccade of the observer is directed to the single particular image. In even other particular aspects of the functions of the method, determining whether the observer had the orienting response and/or the defensive response to the plurality of stimuli includes: determining whether the observer had the defensive response based on a response to the plurality of stimuli. In particular of the even other particular aspects of the functions of the method, the plurality of stimuli include a plurality of images, where a single particular image of the plurality of images is associated with the particular information, and where determining whether the observer had the defensive response includes determining whether the observer made at least one eye movement to a location away from the single particular image. In more particular of the even other particular aspects of the functions of the method, presenting the plurality of stimuli includes presenting the plurality of images in locations with respect to a location of a fixation marker, where the location away from the single particular image includes the location of the fixation marker. In yet other particular aspects of the functions of the method, providing the output of the computing device based on the likelihood that the observer is aware of the particular information includes: determining whether the likelihood that the observer is aware of the particular information is above a threshold likelihood; and after determining that likelihood that the observer is aware of the particular information is above the threshold likelihood, providing an output of the computing device recommending further screening of the observer. In additional particular aspects of the functions of the method, providing the output of the computing device based on the likelihood that the observer is aware of the particular information includes: determining that the likelihood that the observer is aware of the particular information is not above a threshold likelihood; and after determining that the likelihood that the observer is aware of the particular information is not above the threshold likelihood, providing an output of the computing device indicating the observer likely does not have the particular information and so recommending no additional screening of the observer.

In particular aspects of the tangible computer-readable medium, the tangible computer-readable medium is a non-transitory computer-readable medium.

In yet another aspect, a computing device is provided. The computing device includes: means for presenting a plurality of stimuli to an observer, where at least one stimulus of the plurality of stimuli is associated with particular information; means for obtaining at least one measurement of the observer responding to the plurality of stimuli using sensing means associated with the computing device; means for making a determination whether the observer had an orienting response and/or a defensive response to the plurality of stimuli based on the at least one measurement; means for determining a likelihood that the observer is aware of the particular information based on the determination whether the observer had the orienting response and/or the defensive response; and means for providing an output based on the likelihood that the observer is aware of the particular information.

In particular aspects, the plurality of stimuli include visual stimuli. In more particular aspects, the plurality of stimuli include a plurality of images. In other particular aspects of the functions of the method, the at least one measurement includes a measurement of eye movement of the observer. In still other particular aspects, the means for making the determination whether the observer had the orienting response and/or the defensive response to the plurality of stimuli include means for determining whether the observer had the orienting response based on an initial response to the plurality of stimuli. In particular of the still other particular aspects, the plurality of stimuli include a plurality of images, where a single particular image of the plurality of images is associated with the particular information, and where the means for determining whether the observer had the orienting response based on an initial response to the plurality of stimuli include means for determining whether an initial saccade of the observer is directed to the single particular image. In even other particular aspects, the means for determining whether the observer had the orienting response and/or the defensive response to the plurality of stimuli includes means for determining whether the observer had the defensive response based on a response to the plurality of stimuli. In particular of the even other particular aspects, the plurality of stimuli include a plurality of images, where a single particular image of the plurality of images is associated with the particular information, and where the means for determining whether the observer had the defensive response include means for determining whether the observer made at least one eye movement to a location away from the single particular image. In more particular of the even other particular aspects, the means for presenting the plurality of stimuli include means for presenting the plurality of images in locations with respect to a location of a fixation marker, where the location away from the single particular image includes the location of the fixation marker. In yet other particular aspects, the means for providing the output based on the likelihood that the observer is aware of the particular information include: means for determining whether the likelihood that the observer is aware of the particular information is above a threshold likelihood; and means for, after determining that likelihood that the observer is aware of the particular information is above the threshold likelihood, providing an output recommending further screening of the observer. In additional particular aspects, the means for providing the output based on the likelihood that the observer is aware of the particular information include: means for determining that the likelihood that the observer is aware of the particular information is not above a threshold likelihood; and means for, after determining that the likelihood that the observer is aware of the particular information is not above the threshold likelihood, providing an output indicating the observer likely does not have the particular information and so recommending no additional screening of the observer. In still additional particular aspects, the computing device is configured to be operated in a kiosk.

### BRIEF DESCRIPTION OF THE DRAWINGS

FIG. 1 depicts an example contextual placement of an autonomous screening system for the detection of concealed information

FIG. 2 is an example depiction of an orienting response.

FIG. 3 is an example depiction of a defensive response.

FIG. 4 illustrates an example fixation marker and an example stimuli set display.

FIG. 5 shows an example mock improvised explosive device (IED).

FIG. 6 shows an example face stimuli set.

FIG. 7 shows example receiver operating characteristic (ROC) curves for oculomotor defensive behavior.

FIG. 8A shows a photograph of an example mock IED built during a study.

FIG. 8B shows a photograph of an automated screening kiosk.

FIG. 8C illustrates an example slide used in the automated screening.

FIG. 9A illustrates an example kiosk.

FIG. 9B illustrates an example environment where multiple kiosks are operating simultaneously.

FIG. 9C illustrates an operator interface to a kiosk.

FIG. 10 is a block diagram of an example computing network.

FIG. 11A is a block diagram of an example computing device.

FIG. 11B depicts an example cloud-based server system.

FIG. 12 is a flow chart of an example method.

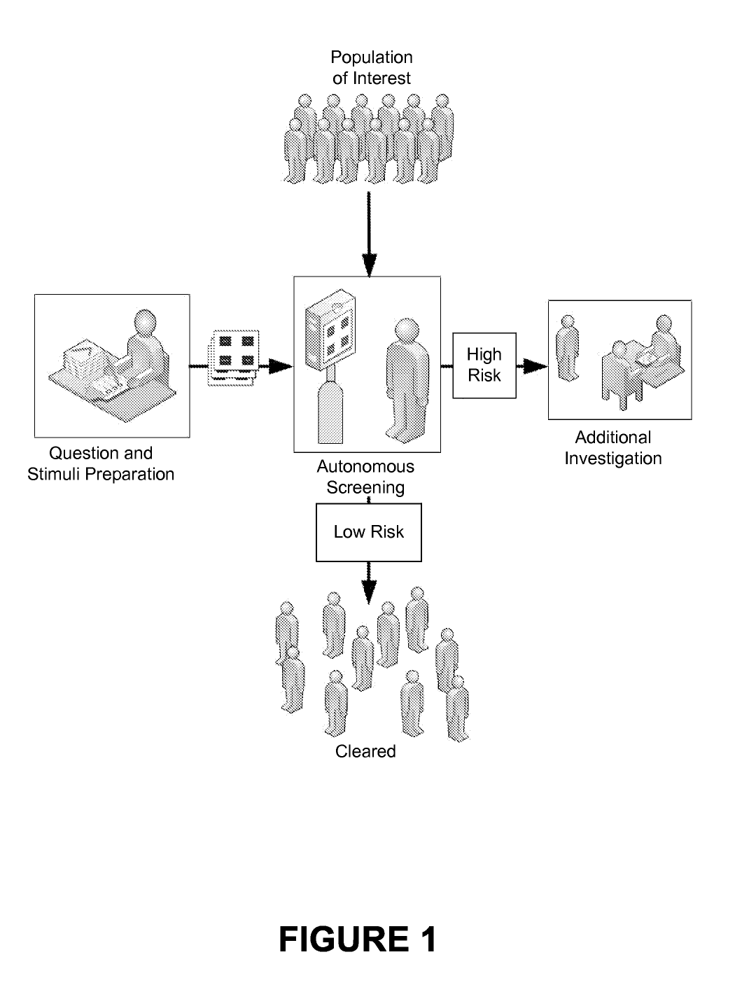

### DETAILED DESCRIPTION

The most difficult type of information to obtain is often that which is intentionally concealed. Yet concealed information is often the most valuable. The perceived ability to conceal information successfully motivates individuals to hide poor performance, commit fraud, conceal system intrusions, or even engage in acts of terrorism. Some high-profile examples include hedge fund manager Raj Rajaratnam, whose covert insider trading generated nearly $54 million in illegal profits. For decades, Bernie Madoff successfully concealed that his financial service was secretly a Ponzi scheme—resulting in a loss of more than $64 billion. No one on board the aircraft knew Umar Farouk Abdulmutallab had smuggled explosives aboard, an act that nearly cost the lives of 289 people.

Information can be hidden by deception, which can include one or more actions taken by a deceiver intended to foster false beliefs or perceptions in a receiver that can occur many forms. For example, it can take the form of exaggeration, white lies, equivocation, complete fabrications, or impersonation. Theories describing deception and its effects explain and predict behavioral and physiological behavior differences between truth tellers and deceivers. To the extent these differences are reliable, exclusive indicators of deception in a given context, detecting deception should be possible.

The detection of concealed information in these and similar circumstances can be thought of as an information credibility problem. Because of the high stakes involved and the breadth of potential applications, the credibility of human-generated information is an important interdisciplinary issue. Many scenarios exist in which the detection of purposely concealed information is the goal when judging credibility. For instance, detecting financial fraud usually involves searching for deliberately concealed data or omitted facts. Criminal forensics and criminal investigations often include searches for evidence that was deliberately hidden. Recruiters desire to discover hidden malicious intentions and past criminal activity in potential employees. Managers are interested in discovering policy noncompliance among employees who are motivated to hide noncompliant behavior. Large-event planning and management personnel need methods of screening people for potential security threats. However, commonly used systems that are leveraged for the detection of concealed information (e.g., the polygraph-assisted “lie detector” test) are costly, time-consuming, and lack scientific support, hindering their application and impact.

This context is an area where information systems (IS) research can make a major impact because there is broad potential application for systems that identify when a person is concealing information. To that end, a foundational, high-level conceptual design for a new class of systems is disclosed termed autonomous scientifically controlled screening systems (ASCSS). ASCSS are unique in that they use highly structured and controlled interviews to assess credibility by detecting the presence of concealed information. Herein is described ASCSS design principles and an ASCSS concept instantiation, a prototype system termed the automated screening kiosk (ASK). Evaluation of the ASK system provides unique insight into orienting and defensive response theories in an automated screening environment.

### Example Design Guidelines for ASCSS

In developing a foundation for a new class of systems for the detection of concealed information (i.e., ASCSS), high-level functional, performance, and process guidelines were identified that focused on credibility assessment. A number of technologies were investigated in combination with various interviewing techniques in a first study. The first study involved a realistic mock crime in which various technologies collected oculometric, kinesic, vocalic, cardiovascular, and thermal data relevant to veracity assessment while participants were interviewed by a professional polygraph examiner who implemented several interviewing techniques.

### ASCSS Functional and Performance Guidelines

Credibility assessment research frames concealed information as a deception problem, where deception can involve concealing correct information through misdirection, omission, fabrication, or another deception tactic. Deception is therefore a manipulation of the integrity of transmitted messages, in part to conceal important information.

Several social psychology theories of deception are built on a leakage hypothesis, which predicts liars would experience abnormal arousal and affect and unintentionally “leak” deception indicators. These indicators can manifest particularly in the hands, legs, and feet. Liars may relieve their tension and discomfort through nervous movements and adaptors (e.g., foot tapping, touching of face). In addition to arousal-induced behaviors, the leakage hypothesis predicted that liars will also inhibit certain behaviors and natural gesturing.

The Four-Factor theory added increased cognitive effort and overt behavioral control as sources of leakage to the leakage hypothesis. That is, liars not only are predicted to exhibit leakage indicators due to arousal and negative affect, but also experience more cognitive load while managing their lie and appearance. This increased vigilance in appearance also causes over-control of normally automatic and natural interaction gesturing, causing rigid and inhibited behavior. If a deceiver does not experience fear or arousal, then cognitive or behavioral control indicators could be more diagnostic.

Interpersonal Deception Theory frames deception within the context of an overarching interaction. Depending on the skill of the sender and their relationship with the receiver (e.g., boss, parent, loved one), deceivers are predicted to employ dynamic strategies in their effort appear credible. A liar may start out feeling confident and reveal few behavioral indicators of deception, but after sensing suspicion, begin compensating and exhibiting different indicators and gestures.

Cognitive psychology and psychophysiological deception detection are at the heart of the polygraph and similar deception detection techniques. These practices draw on theories describing the psychophysiological orienting of attention and defensive response (traditionally termed a “fight or flight” response) that is expected to occur when a deceiver is presented with a stimulus (e.g., a question or an image) that is perceived as personally significant and threatening. When attention orients toward a personally significant stimulus, measurable physiological changes occur, such as changes in pupil dilation and skin conductance. If that stimulus is perceived as threatening, a defensive response also occurs, which can transition to defensive behaviors such as those outlined in social psychology literature.

Perhaps the most common system used for assessing message credibility is the polygraph. A polygraph system measures cardiorespiratory and skin conductance data through sensors attached to a human examinee and tracks these signals over time. Some interrogation methods employ polygraph data as a decision aid, leading to deception detection results about 15 to 30 percentage points greater than unaided human performance, which generally hovers around 54%.

However, the polygraph and common interrogation methods using the polygraph are riddled with limitations that prevent application beyond criminal investigations and similar policing activities. Common polygraph-assisted interviews often require hours to complete and additional time to analyze. Polygraph sensors are invasive and require training for proper attachment and calibration. The data from a polygraph are presented in raw fashion, leaving room for subjective interpretation of results. These and other limitations discussed in subsequent sections make use of polygraph systems infeasible for organizational credibility assessment needs, such as policy compliance assessment, job application fraud detection, physical security screening, insider threat detection, and similar applications in which credibility decisions are made rapidly and professional deception detection skill is uncommon.

In such scenarios, screening for concealed information needs to be conducted rapidly, and technologies and techniques for detecting knowledge cannot require invasive sensors or extensive calibration. A system capable of conducting an interview and generating an assessment autonomously would minimize the need for professional skill, decrease subjectivity, and increase efficiency by automatically filtering out persons who are a low risk.

Prior observations identified other key functional and performance guidelines. As participants were being interviewed regarding their potential involvement in the (mock) crime, noninvasive oculometric, kinesic, vocalic, linguistic, and cardiorespiratory sensor data were collected. Each sensor showed promise for credibility assessment under certain conditions. However, some technologies required screening protocols that were quite lengthy and not easily automated, and other sensors functioned poorly with an unacceptably high percentage of screening candidates. If automation is a central component of a screening system, the technology-technique combination used should be applicable to a high percentage of screening candidates.

An initial investigation also illuminated important interaction constraints. The polygraph examiners exhibited major differences in interviewing style, demeanor, and approach, in spite of the fact that each followed a semi-structured script. The variations in interaction style likely affected the generalizability of the results. For instance, one interviewer chose a calm, rapport-building approach; another chose a more belligerent, accusatory tone. In differing interviewing styles, different cues to concealed-information deception might be expected. It is possible that other factors such as interviewer gender, skill level, and types of follow-up questions can affect the type and intensity of deception indicators. These observations suggested that a highly controlled, standardized interaction with a predefined questioning protocol may be desirable.

From a performance standpoint, the first point of comparison is unaided deception detection, which a meta-analysis has shown to be around 54%, regardless of professional status or confidence level. Polygraph-assisted screenings are often compared to and have been shown to exceed this standard, and the same standard should apply to an autonomous hidden-information risk assessment system: The system's abilities can exceed those of an unaided professional. This feat was accomplished by several noninvasive sensor technologies in the first study. Table 1 summarizes the functional and performance-related guidelines identified as a result of the initial investigation.

TABLE 1
Functional and Performance Guidelines for Autonomous Human
Screening for Concealed Information

| # | Guideline Description |
|---|---|
| 1 | Conduct an interview autonomously. |
| 2 | The sensing technology used cannot be invasive or require extensive |
| | calibration. |
| 3 | Employ sensing technology that operates effectively with a large |
| | percentage of human examinees (95% or greater, depending on |
| | context). |
| 4 | Conduct a standardized interview that is consistent for all persons of |
| | interest. |
| 5 | Perform the interview rapidly, requiring only a few minutes or less |
| | per examinee (precise time constraints will be application-specific). |
| 6 | Generate a risk score assessing the likelihood that a given examinee |
| | is purposely concealing information about a target topic of interest. |
| 7 | Identify concealed information with an overall accuracy rate greater |
| | than unaided human judgment. |

### ASCSS Interview Protocol Guidelines

As indicated above, a standardized and consistent interview can be important. This also distinguishes ASCSS from other types of deception detection systems in IS literature. Whereas structured interaction techniques are a staple in criminal justice and forensic psychology research, IS deception detection research has almost exclusively examined deception in natural or unstructured interactions, including online chat, written statements, data evaluation, e-commerce interactions, virtual interactions, credibility assessment decision-support with computer aids, or open-ended interviews. Because cues to deception and concealed information are influenced by many factors, deception detection algorithms cannot be applied equally to every situation. Psychology and criminal justice research on deception detection demonstrates that when it comes to screening, the protocol, or the procedures used to assess veracity, is just as important as the observed veracity indicators or the measurement tools. There is no indicator of deception like a “Pinocchio's nose” that is highly accurate regardless of context. Quite the opposite, the effectiveness of a tool that assesses veracity is influenced by factors such as interviewer skill, and crime-related knowledge communication synchronicity, and even the type of questions asked. It was thus important to identify reliable and generalizable protocol guidelines in which these contextual variables are controlled.

Several credibility assessment questioning techniques in forensic psychology research were investigated to identify potential protocol guidelines. The current state of this area of research helps establish the value of a standardized screening protocol. Several standardized interpersonal screening techniques are regularly used—the most common of which include the comparison question test (CQT), the behavioral analysis interview (BAI), and the concealed information test (CIT).

Among practitioners, the CQT is currently the most commonly used interviewing technique for credibility assessment. The CQT takes several hours to complete, and requires a high level of interviewer skill to obtain valid results. Though the CQT is commonly used in practice, some criticize the test as having a weak theoretical foundation. Several questions mimicking the CQT format were included in the first study. The CQT line of questioning showed some potential for identifying concealed information. However, prevalent in this technique was a heavy reliance on dynamic follow-up questioning, open-ended responses, and the potential for question type effects that introduce uncontrolled factors.

The BAI is a method of interviewing that is probably the second most common interviewing technique used in the United States. Some direct investigations have challenged the BAI's validity though the ecological validity of these studies has been called into question. The mechanisms underlying the BAI remain underexplored, and much more research is needed before the validity of the BAI can be established.

The CIT is not used as commonly by practitioners as are the CQT or BAI. Unlike these more common techniques, the CIT does not rely heavily on the interviewer's capabilities. Instead, the CIT interviewer plays only a minor role—requiring little to no skill. Though the CIT is not as commonly used as the CQT or BAI, researchers widely consider the CIT the most scientifically valid approach. The CIT requires the least time and little interviewer skill or intervention—features that not only help to control for interviewer effects but also make automation feasible. Evaluators can complete several question sets in a matter of minutes, whereas alternative techniques can last for hours. Existing research has shown that the CIT clearly has value when more invasive sensors are used, such as electrodermal activity sensors or functional magnetic resonance imaging (fMRI). The first study investigated several types of noninvasive sensors in identifying concealed information via the CIT protocol. Table 2 summarizes the structured interviewing methods.

TABLE 2
Structured Interviewing Methods Used for Detecting Concealed Information

| | | Scientific | Common | Interviewer | |
|---|---|---|---|---|---|
| Interview | Time | Consensus | Criterion for | Skill Level | Practitioner |
| Technique | Requirement | on Validity | Assessment | Required | Usage |
| CQT | 2-4 | Low | Elevated arousal | High | Widespread use |
| | hours** | validity | | | in North America, |
| | | | | | Asia, and Europe |
| BAI | 15-45 | Uncertain; | Expert analysis | High | Used in the |
| | minutes*** | nuanced | of verbal and | | U.S. and some |
| | | | nonverbal behavior | | international |
| | | | during interview | | use, including |
| | | | | | some business |
| | | | | | applications |
| CIT | 2-15 | High | Elevated orienting | Very low | Limited to |
| | minutes* | validity | response | | Japan and |
| | | | | | some use in |
| | | | | | Israel |

*Exact time is a function of how many questions are used (usually between 3 and 6).
**Estimated from a subjective review of polygraph examiner practitioner promotional material.
***Lower-bound estimate based on amount of time required to minimally ask and respond to all BAI questions. Upper-bound estimate reflects the potential for follow-up questions.

Because of success using the CIT in the first study, scientific consensus on the protocol's validity, its brief interaction time, and its strict control, principles underlying the CIT warranted a closer examination for application to ASCSS. In a standard CIT, a human interviewer recites several prepared questions or statements regarding the activity (e.g., crime) in question to a human examinee. Created for each question are several plausible answers, collectively called a foil, which are also recited by the interviewer. For instance, if the activity in question is the theft of a vehicle, one of the CIT statements might read, “If you were involved in the theft of the vehicle, you would know the color of the car that was stolen. Repeat after me these car colors.” The interviewer would then verbally recite each item in the associated foil, which would consist of about four to six names of colors; one of these would be the correct color (i.e., target foil item). The examinee is usually asked to either repeat the items or reply with a verbal “yes” or “no” after each item is spoken by the interviewer. Once the examinee has spoken, the examinee and interviewer sit in silence for several seconds while psychophysiological measurements are recorded.

The low ratio of relevant to non-relevant options within each foil (usually between 1:4 and 1:6) allows for a strong person- and question-specific baseline. As a result, indicators of concealed information are not easily attributed to outside factors such as overall anxiety or uncertainty about being interviewed, differences in interviewing style, or interviewer demeanor or skill. Each foil is self-contained; a system or evaluator can use a single foil to make a judgment. However, using multiple foils reduces the probability of false positives as long as each foil is central to a common topic of interest.

### ASCSS Measurement Guidelines

The psychophysiological and behavioral processes that can be leveraged using ASCSS can be evaluated to determine which human signals and technology sensors can serve as effective measurement tools. These psychophysiological and behavioral processes can include processes involving an orienting response and and/or a defensive response. Herein is disclosed how each response can create measurable indicators of concealed information in a highly controlled interviewing protocol.

### Overview of the Orienting Response

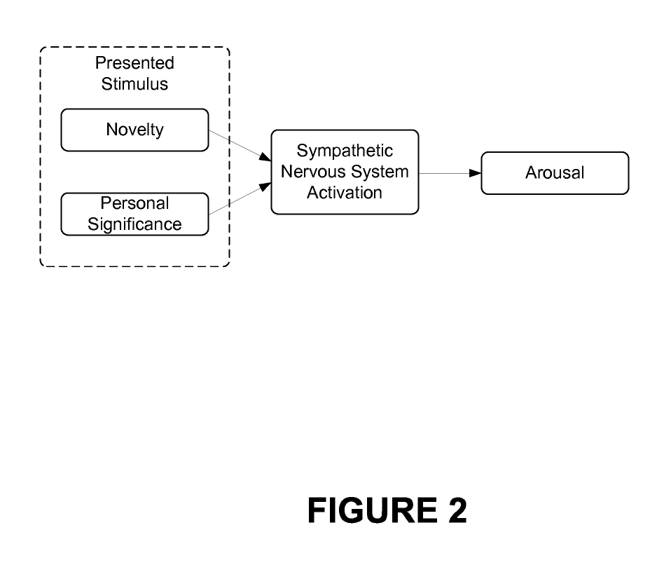

The orienting response (or reflex) is the autonomic movement of attention toward novel or personally significant stimuli. The level of stimulus novelty is a function of the degree to which the stimulus matches (or does not match) stimuli that precede it in a given context. The level of personal significance is a function of the degree to which a stimulus matches one's cognitive representation of a given item of relevant information. When an individual's autonomic system registers a novel or personally significant stimulus, the sympathetic portion of the nervous system activates to mobilize the body to a state of readiness (i.e., arousal) so that the individual is ready to adapt or react to the stimulus. This transition to a readiness state includes physiological changes such as variance in heart rate, skin sweatiness, pupil dilation, and respiration. Stimuli that have stronger personal significance or “signal value” such as an out-of-place object or hearing one's own name produce a stronger orienting response. With repeated presentations of stimuli, the magnitude of the response decreases as a function of the corresponding decrease in novelty and personal significance. FIG. 2 depicts an example process of activating the orienting response.

### Overview of the Defensive Response

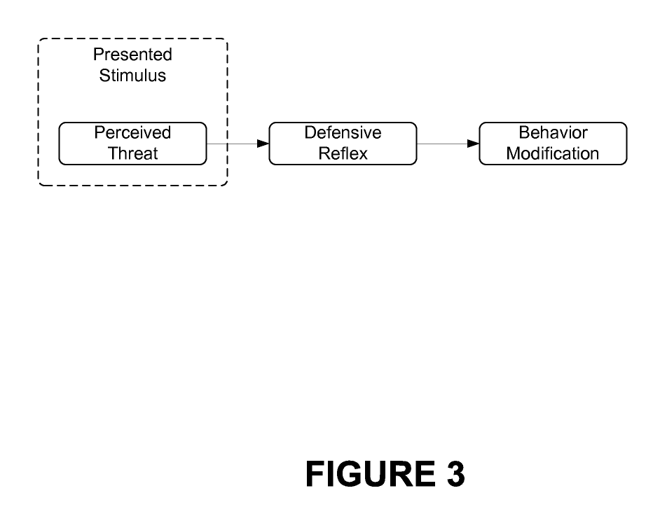

FIG. 3 depicts the defensive response to a perceived threat. Though the defensive response has received less attention in CIT research than the orienting response, defensive behaviors have been the focus of much research in credibility assessment literature and are key indicators in less structured interviewing techniques. Although the orienting response can occur with any stimulus of sufficient novelty or personal significance, the defensive response is a reaction only to stimuli perceived to be aversive or threatening. This reaction includes physiological and behavioral changes.

The defensive response was initially coined the “fight-or-flight” behavior in the early 20th century. The defensive response can be broken down into at least two phases—(1) an initial defensive reflex (2) followed by defensive behaviors. When threatening stimuli are first perceived, the sympathetic nervous system is activated—driving a defensive physiological reaction thought to help the individual assess the threat and determine the appropriate action to take. Many of the physiological changes associated with this initial defensive reflex are similar to the orienting response (e.g., a sudden increase in skin sweatiness), though differences are manifest in the cardiovascular response.

The initial defensive reflex transitions into behaviors designed to escape or combat the threat. In this context, a term of defensive behaviors can be used to distinguish such responses from the defensive reflex. Behaviors that stem from responding to a threat are not necessarily autonomic and can be driven by either subconscious or conscious mechanisms. Defensive behaviors are driven by a perceived threat and therefore can be different from behavioral reactions to stimuli perceived to be non-threatening. Credibility literature has documented various “fight-or-flight” tactics individuals consciously or subconsciously employ when an important deception is under threat of discovery.

In CIT research, the response stage of the defensive response is thought to amplify many of the physiological measures of the orienting response. Defensive behaviors have not been investigated in CIT research, but their identification in more natural or semi-structured deception detection interactions suggests that they can have potential in a highly structured interview as well. Stimuli that have the potential to expose concealed information about a topic that an individual wishes to keep hidden should trigger defensive behaviors designed to escape or combat that perceived threat. The same stimuli should have no such effect on individuals who are not concealing information for they should not find them any more threatening than non-relevant stimuli. Behavior modifications can consequently reveal the presence of purposely hidden information in a CIT-like interview format.

Within an ASCSS protocol framework, both orienting and defensive behaviors can generate measurable indicators of concealed information. The protocol design of ASCSS calls for multiple choice-type questions, with only one correct answer per question. The correct answer should create additional “signal value” for those being screened only if they place particular personal significance on that item above and beyond alternatives. This structure allows physiological and behavioral measures that follow the presentation of a relevant stimulus to be compared to the same measures following the presentation of irrelevant stimuli. For instance, the cardiovascular interbeat interval (IBI) measure tends to be abnormally long following responses to relevant versus irrelevant items. Whichever indicators of concealed information are used, the protocol of ASCSS will not be used to its full advantage unless recorded data are standardized on a person-specific and question-specific baseline. Table 3 overviews the measurement and protocol guidelines derived for ASCSS.

TABLE 3
ASCSS Interview Protocol and Measurement Guidelines

| # | Protocol and Measurement Guideline |
|---|---|
| 1 | Present predefined questions or statements based on credibility topics |
| | of interest. |
| 2 | For each question/statement, present multiple stimuli as possible |
| | answers. |
| 3 | Only one stimulus should represent the target topic of interest. Other |
| | stimuli should be presented as similar but conceptually distinct. |
| 4 | During presentation of each stimulus, automatically capture human |
| | indicators of the orienting and/or defensive responses. |
| 5 | Evaluate responses in a manner that controls for interpersonal baseline |
| | differences. |

### An Example ASCSS Instantiation: The Automated Screening Kiosk (ASK) System

To further define and establish the concept of an autonomous screening system for hidden information risk assessment, the ASK system was constructed as a specific implementation of the ASCSS design guidelines. The ASK system was designed to conduct a rapid, structured interview automatically while collecting oculomotor (i.e., eye movement) data. The ASK system collects eye-movement data using an EyeTech™ eye tracking device. The system requires a preparation phase in which target topics of interest and stimuli sets representing instances of these topics are identified. The ASK system accepts stimuli sets in the form of images paired with pre-recorded questions. The number of stimuli sets displayed and the length of time a given set is displayed are configurable as they are expected to vary based on application-specific guidelines.

Once the ASK system has received visual and audio input, the system waits in a readiness state until the ASK system recognizes eyes within the field of recognition of the eye tracking sensor. At this point, a computer-generated voice gives initial instructions to the person being screened. The ASK system then guides the individual through a 10- to 15-second calibration process, which allows the device to more accurately track each individual's unique oculomotor activity.

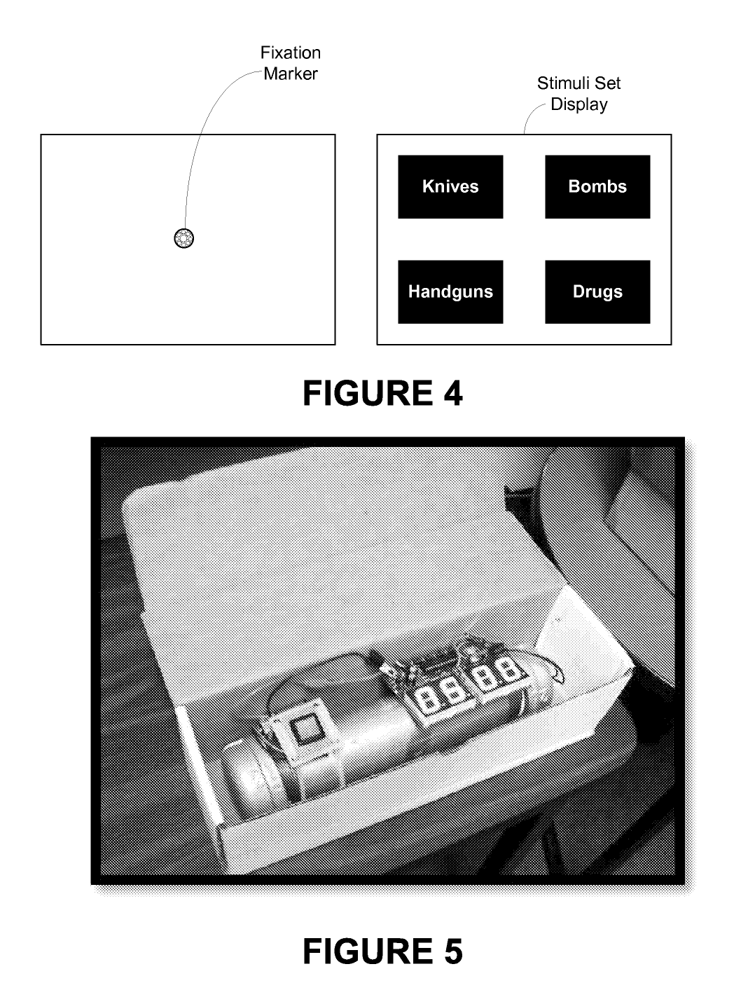

Following a successful calibration, the ASK system asks structured questions paired with stimuli sets following the script configured at the beginning of the process. While a given question is asked, the screen remains blank except for a fixation marker in the center of the screen. This fixation marker standardizes the starting point for visual attention before a foil is presented. The fixation marker also serves as the single point on the screen equidistant from all foil items (the “safest” place). An example fixation marker is depicted in the center of a left-side image of FIG. 4.

Immediately following each question, the fixation marker disappears and the ASK system displays a stimuli set consisting of four boxes on the screen that are equidistant from one another and from the screen center. An example stimuli set display is depicted on the right-side image of FIG. 4. These stimuli sets are displayed for a short (configurable) duration, allowing time for the participant to examine each stimulus and respond to the question verbally with “Yes” or “No.” Raw oculometric data are collected at approximately 30 Hz and are automatically tagged with the associated stimuli screen.

An entire screening process takes approximately two minutes, assuming four to five stimuli sets and a seven-second presentation period for each. After completing the process, the ASK system instructs the participant to proceed and then returns to a readiness state, awaiting the next candidate. These features allow autonomous system operation, automatically clearing all candidates and flagging only cases where the ASK system determines that further screening is desirable. In such circumstances, the ASK system can alert a managing human agent while simultaneously directing the traveler to a secondary screening station. In this proof-of-concept iteration of the ASK system, categorization algorithms used a novel oculomotor defensive behavior measure as the decision criterion.

### Measuring Orienting Behavior via Eye-Movement Tracking

To meet the measurement guideline for noninvasive concealed information indicators, ASK leverages the visual stimuli sets to capture novel measures of orienting and defensive behavior using oculomotor (eye movement) tracking. Traditional measures of the orienting and defensive responses target skin conductance response (SCR), respiration, and heart rate. Traditional sensors for measuring these physiological reactions require direct contact and manual calibration and supervision. These considerations prompted an evaluation of alternative measures for detecting concealed information. Some CIT research includes using functional magnetic resonance imaging (fMRI) or similar brain imaging techniques, but the procedures and measurement apparatus for these scenarios are even more invasive than traditional techniques and would require even more specialized supervision. Eye movement patterns can betray deception, and a second study revealed potential for oculometric indicators of concealed information. For security screening, eye-movement tracking can be leveraged as a noninvasive, automated alternative for measuring orienting and defensive responses.

When a presented novel or significant stimulus demands visual processing, the eyes reflexively orient toward the stimulus. The rapid movement of the eye from one point of visual focus to another is termed a saccade. Saccades are the most common type of eye movement, and can be reflexive, such as when driven by the orienting response, or overt, such as when performing a visual search task.

Eye-movement patterns have long been used in cognitive psychology research to explore the visual orienting response and overt attention shifts. Some neuro IS researchers have similarly begun to use eye movements as surrogates for visual attention. The spotlight theory of attention posits that stimuli outside the focus of attention are processed by peripheral attention. Visual stimuli are first discovered by peripheral attention; if a stimulus has a sufficient level of significance or novelty, the eyes move toward the stimulus. Saccades can be either reflexive or overt (i.e., consciously controlled). To the extent saccades are reflexive, they will occur before the stimulus is identified consciously.

The ASK system is uniquely designed to exploit this reflexive visual orienting. The ASK system uses visual rather than auditory CIT foils and presents foil items simultaneously rather than in a sequence. If visual stimuli are displayed simultaneously on a screen, like the stimuli set display illustrated in FIG. 4, those who are hiding knowledge about a particular event should be more likely to orient their initial attention reflexively, and therefore their eyes will orient toward the visual CIT item associated with their guilty knowledge. For instance, if a visual CIT foil consists of the words “bombs,” “knives,” “guns,” and “ammunition,” a person hiding an explosive device should reflexively saccade toward the word “bombs,” as this word would have the highest level of personal significance relative to the alternative items. In contrast, a person without guilty knowledge would be significantly less likely to orient toward the word “bombs.” Again, orienting theory posits that over time the orienting response diminishes in a manner corresponding to the associated decrease in novelty and/or personal significance. As individuals gain experience with the format of a rapid screening CIT, they could find the novelty of the stimuli diminish.

### Measuring Defensive Behavior via Eye-Movement Tracking

In some cases, upon initially detecting the critical foil item, persons with guilty knowledge can exhibit defensive behavior. Though several possible autonomic or overt actions can be taken, defensive response theory holds that the default behavior is usually avoidance or escape. The simultaneously presented visual CIT stimuli design takes advantage of this phenomenon: The ASK system presents a “safety” point in the center of the screen when each question is recited audibly. The safety point's position is equidistant from all visual stimuli—serving as the optimal point of avoidance or point of greatest safety away from potential threats. In some cases, examinees with hidden guilty knowledge can focus more visual attention on the best point of escape—the center point of the screen. Those without guilty knowledge should manifest significantly less propensity to orient to this center point because they will not share this inherent propensity for avoidance.

In summary, eye gaze is hypothesized to initially orient toward stimuli associated with concealed information; then, as a potential threat is noticed, gaze should show a tendency to defensively avoid stimuli. Unlike the orienting response that diminishes with time, defensive behavior should remain constant as long as the examinee has reason to perceive a threat. Namely, a credible threat yesterday does not diminish another credible threat today. This consideration is important for contexts such as security screening in which testing can occur several times for frequent travelers/entrants. Table 4 outlines predicted outcomes if the ASK system simultaneous stimuli sets design is successful.

TABLE 4
Expected Empirical Outcomes of ASK's Oculomotor Cues to
Concealed Information

| Behavior | |
|---|---|
| Measurement Type | Expected Oculomotor Outcome |
| Orienting response | H1. Concealed information will increase the |
| | likelihood that an initial saccade will be directed |
| | toward the target item in a collection of |
| | simultaneously presented CIT foil items. |
| Orienting response | H2. With repeated exposures, stimuli representing |
| | concealed information will be less likely to attract |
| | the initial saccade. |
| Defensive response | H3. Concealed information will cause increased |
| | stimuli avoidance when the target item is present. |
| Defensive response | H4. Concealed information will cause stimuli |
| | avoidance even during repeat screenings. |

### Facial Movement, Pupil Dilation, and Kinesic Rigidity Measures

Other more physiological measurements, such as facial movement, pupil dilation, and/or kinesic rigidity measurements, can be observed to detect defensive and/or orienting responses as well. For example, facial movement, such as facial expressions can be used to detect orienting and/or defensive responses. For example, a person may grimace, snarl, or otherwise reflect unpleasantness on their face as a defensive response when confronted with one or more stimuli related to concealed information. Further, a person may look intentionally bland or otherwise act dispassionately as an orienting response while remaining in the presence of one or more stimuli related to concealed information.

Changes in pupil dilation can be linked with a number of cognitive functions. Preliminary research discovered that changes in pupil dilation can be used to identify activation and arousal in autonomic activity. Additionally, differences in pupillary responses have been linked with short-term and long-term memory retrieval. A study investigating differences in pupil dilation associated with viewing novel and repeated stimuli revealed that a pupil exhibits increased dilation when repeatedly exposed to a given stimulus, referred to as the Pupil Old/New Effect, or PONE.

Pupil dilation has been shown to be part of the orienting response. The orienting response is traditionally a key human factor of interest in a CIT, and tracking electrodermal activity (skin sweatiness) is the standard method for measuring the orienting response. However, the physiological activation triggered by the orienting response also triggers several other physiological changes, including pupil dilation. Within CIT research, evidence supports the notion that common mechanism triggers both pupil dilation and electrodermal activity in response to concealed knowledge. Additionally, changes in pupil dilation during a CIT are attributable to simply having concealed knowledge and not necessarily to any concurrent deceptive behaviors, such as verbally lying. The use of pupil dilation as a cue to deception is dependent on the differential response to the target item. The arousal triggered by recognition of the target item causes the pupil to dilate to a much greater extent than when viewing other items.

Countermeasures

Countermeasures have been shown to have significant impact on polygraph tests and brain imaging tests. Traditional countermeasures function primarily by manipulating the participant's responses in a manner that is expected to minimize the difference between baseline (i.e., truthful) responses and responses to questions or other stimuli that may result in deception.

Countermeasures fall primarily into two categories: mental and physical. Physical countermeasures are deployed using a variety of behaviors including finger movements, pressing toes against the floor, and biting the tongue. These physical countermeasures are employed during the portion of an interview that is designed to capture baseline physiology. By mimicking a physiological response (e.g., generating pain will induce arousal) during baseline items, examinees may effectively obfuscate their deception if evaluations reveal no significant difference between baseline and deceptive responses.

Mental countermeasures can also be used to try and mimic physiological responses, but more often their goal is to suppress such responses through mental distraction. Mental countermeasures include silent counting, recalling past emotional events, or distractions such as simply mentally reciting one's own first and last name. Mental countermeasures are either employed during the baseline portion if the goal is to mimic, or during the entire interview if the goal is to suppress. A polygraph-based study found that counting sheep throughout the length of the test decreased detection rates. A P300-based deception detection experiment showed that participants who mentally recited their first and last name during two of four baseline stimuli were able to modify their responses enough to evade detection.

Where countermeasures are shown to be effective at manipulating deception detection results, cognitive psychology research has turned to detecting countermeasures. Physical countermeasures are especially vulnerable to detection. In some cases, 90% of countermeasure users could be identified using an electromyograph to measure muscle activity in the legs and head. Similarly, while slight finger movements reduced detection accuracy in fMRI-based tests, they also increased activation of the motor cortex, the part of the brain responsible for movement. Increases in reaction time allowed countermeasure detection in P300 mental countermeasures.

The employment of mental countermeasures to artificially increase pupillary response to non-target items could help artificially raise the baseline of comparison, resulting in a less accurate determination of deception. Because pupil dilation is related to cognitive processing, taxing mental tasks can increase pupil dilation on demand. By performing mental arithmetic during non-target items, the pupillary response during the target item can thereby be masked.

The following predictions can be made regarding pupillary responses and target items:
(1) Deceptive individuals will have a larger pupillary response to target items than to non-target items,
(2) Deceptive individuals using mental countermeasures will exhibit reduced pupil dilation differential between target and non-target items compared to deceptive individuals using no countermeasures, and
(3) Physical countermeasures will reduce the pupil dilation differential between target and non-target items.

The employment of mental countermeasures based on cognitive function to artificially increase pupillary response could help artificially raise the baseline of comparison, resulting in a less accurate determination of deception. Because pupil dilation is also related to cognitive processing, taxing mental tasks may increase pupil dilation on demand. If an examinee is to respond plausibly, mental countermeasures performed by the examinee are unlikely to be distracting enough to avoid an autonomic orienting response to provided stimuli.

In addition to cognitive effort, pupil dilation can also be triggered by pain. In electrical stimulation experiments, pupil dilation was shown to increase nearly immediately at the onset of pain, and that this dilation increases with increasing pain intensity. Further research has shown that the pupil dilation is not only immediate, but lasts for the duration of the pain. These results indicate that physical countermeasures such as biting the tongue, as employed in polygraph studies, may also work to artificially manipulate the pupil dilation baseline in an automated CIT screening paradigm. As with mental countermeasures, causing pain during non-target items should increase the pupil dilation, reducing the difference between target and non-target pupil dilation in a CIT.

Kinesic rigidity is the constriction of body movement. Kinesic rigidity has been found in communication research to be an indicator of low veracity during open-ended or semi-structured interviewing techniques. When lying, participants tend to exhibit less overall movement, especially expressive or illustrative gestures, and the movement that does occur tends to be spatially constricted and appear forced rather than natural. This phenomenon is also present in the more controlled CIT interview setting, and has developed a method for automatic detection of kinesic rigidity via comparison of body movement during baseline items to body movement during target items.

Likely because of the high cost of traditional measurement of kinesic rigidity, this cue to deception is not used in practice. It has also received almost no attention in countermeasures research. One psychology study determined that controlling kinesic rigidity is very difficult in semi-structured interviews, at least when trying to control it directly. However, there are still many unknowns, including the effectiveness of traditional countermeasures, and how well kinesic rigidity can be overtly controlled in a highly controlled automated screening setting where many behaviors are to be controlled simultaneously.

In semi-structured interviews, kinesic rigidity has been hypothesized to stem from cognitive overload, in that more cognitive effort is being placed on mentally constructing and relaying a plausible story, leaving fewer resources to allocate toward nonverbal presentation, creating less overall movement and more constricted movement. A second theory suggests that kinesic rigidity itself may be a form of countermeasure, in that because people generally falsely believe that liars exhibit increased movement and so purposely minimize their own movement to appear truthful. A third possible explanation is the biologically-driven freeze response that all humans experience when confronted with something that is threatening. Previous IS research discovered kinesic rigidity in a CIT, which requires no communicative or illustrative movement, so cognitive overload will not be a likely driver in the highly controlled, automated format employed by the system design used. It is possible that the root cause is a combination of the freeze response and behavioral control.

The following predictions can be made regarding kinesic rigidity and target items:
(1) Deceptive individuals will exhibit less overall movement when viewing and responding to a target item.
(2) Deceptive individuals employing mental or physical countermeasures will exhibit less overall movement when viewing and responding to a target item.

### Evaluation of the ASK System

The ASK system was evaluated empirically to begin to establish evidence as to whether the ASCSS class of systems can work as effective detectors of concealed information. Because the purpose of this project is to provide evidence toward a proof-of-concept for a concealed information detection system, an appropriate method for evaluating the ASK system prototype was by conducting a laboratory experiment simulating a sample scenario. The experiment involved having participants construct and pack a mock improvised explosive device (IED) and then attempt to bring it through a security screening station.

### ASK Evaluation Experiment Summary

Adult participants (N=134) were recruited from a metropolitan area through flyers, newspaper advertisements, and social media ads to participate in a deception experiment. Those participants who were randomly assigned to the “guilty” condition undertook an elaborate set of actions to commit a mock theft of a “diamond” ring from a secretary's desk in a building down the street. Participants received these instructions from an audio recording of a mechanical-sounding voice. Those assigned to the “innocent” condition engaged in many of the same steps as the guilty participants but did not commit a theft.

Specifically, participants were instructed to go directly to an upper floor in a nearby building and tell the receptionist that they had an appointment with a Mr. Carlson. Participants in the guilty condition were aware that Mr. Carlson did not exist, but were told that the receptionist was new and would have to leave the room to confirm this. These participants were then supposed to steal a diamond ring from an envelope contained within a cash box in the receptionist's desk drawer, conceal the ring on their person, destroy the envelope, and be careful not to leave any fingerprints. They were also required to also make up a cover story in case someone asked them questions or if they were caught. Participants in the innocent simply waited at the door for the receptionist to return.

Upon returning, the confederate receptionist instructed participants to go to another room. Upon arrival, a project staff member met them, asked them some questions, and then escorted them to a credibility assessment test room equipped with many non-contact sensors, where they were interviewed by one of four certified polygraph examiners. The interview consisted of 34 questions, leveraging CQT, BAI, CIT, and startle blink protocols. Polygraph examiners were instructed to follow the script but were allowed to ask follow-up questions on the more open-ended questions to better approximate their normal manner of interviewing.

A number of sensors recorded participant responses. Physiological responses were measured with a laser Doppler vibrometer (LDV) that measured cardio-respiratory responses and with a thermal camera that measured blood flow to the periorbital region of the face. A fast (60 frames per second) near-infrared camera measured pupil dilation and blink responses, and an ultra-fast (250 frames per second) infrared blink camera measured blink intensity during presentation of startle stimuli. Three visible spectrum, high-speed (30 frames per second) digital cameras recorded facial close-ups, full-body images, profile images, and the audio signal. These audiovisual recordings were subjected subsequently to automated and manual analysis of kinesic-proxemic, vocalic, and linguistic behavior. Following the interview, participants' eye movements were tracked with an eye tracking device as they responded to visual stimuli that should have been familiar only to those committing the crime.

In contrast to a standard polygraph interview in which most responses are confined to short answers, or more experimentally constrained interviews with no options for follow-ups or digressions from the script, these interviews utilized an extended battery of questions and allowed interviewers the latitude to ask follow-up questions during BAI and some CQT questions. Greater flexibility came, however, at the expense of experimental consistency.

Some questions elicited very brief responses that were appropriate for measuring physiological responses but possibly too limited to yield enough useful kinesic or linguistic data. Conversely, questions that elicited lengthier responses produced more behavioral data but can introduce error in the search for minute physiological anomalies. Variability in interviewer style and in interviewer prefatory or follow-up remarks added further variability that contributed to measurement error. These problems notwithstanding, the results showed that some physiological and behavior indicators hold promise as noncontact measures of veracity.

Summarizing the relevant results of this experiment, many potentially useful cues to deception were found. However, these anomalous behavioral or physiological changes detected using noninvasive sensors were not always consistent across interviewing styles, test protocols, cultural dispositions, types of deception, and so forth. These results provided initial support for the potential of noninvasive sensors for credibility assessment, but highlighted the potential value of a highly controlled system design (i.e., ASCSS).

### Evaluation Context

Security screening is an optimal context for evaluating the ASK system. A goal of security screening is to identify concealed threats prior to allowing entry. Though sometimes a threat can be easily identified by a metal detector or similar system, many threats are carefully concealed, making detection difficult. Often only a few seconds of screening time are allocated for each individual. Manpower for security is costly and limited by human bias and performance variability. For example, the U.S. Department of Homeland Security (DHS) processed more than 267 million incoming border crossings in 2008 but estimated a 71.1% failure rate when it came to apprehending major violations of laws, rules, and regulations. Air passenger violators were also reportedly apprehended only 25% of the time.

One strategy for greater process efficiency and effectiveness is to perform screening in stages, sending only those deemed by automated systems to be a possible risk to human-driven secondary screening, simultaneously providing greater throughput and more screening time for potentially high-risk candidates. In this layered approach, the first-line system would be an ideal fit for ASCSS because of the need for rapid evaluations and control for variability due to interpersonal differences.

### Experimental Design

The experimental design for the first study involved two treatments with four repeated measures repeated on two subsequent days. The two treatments were guilty and innocent. Half of the participants were randomly assigned to the guilty treatment, which involved constructing a mock IED (i.e., bomb) and packing it in a bag. Innocent participants also packed a bag. Participants in both conditions carried the packed bag through a mock building security screening station. The purpose of constructing an IED was to simulate realistic concealment of information as closely as possible in a laboratory environment. Participants interacted with the screening system twice, with the second interaction being a replication of the first, but occurring on the day immediately following the initial interaction. The repeated measures occurred at the screening station, at which participants viewed four visual CIT foils with oculomotor measurements recorded during the presentation of each foil.

In the second study, each individual also packed a bag as discussed in the paragraph above. However, each individual was randomly assigned to one of the following five conditions: guilty with no countermeasures, guilty with mental countermeasures, guilty with physical countermeasures, guilty with all (mental, physical, and additional countermeasures), and an innocent (control) group. The number of participants assigned to each group was roughly equal. Participants in the guilty conditions were tasked with constructing an improvised explosive device, concealing it in a bag with standard travel items, and delivering the bag to a person in a secure portion of a building. Participants randomly assigned to one of the three countermeasures groups also constructed the improvised explosive device and packed it in a bag; however, they also completed training on how to use countermeasures to defeat the system. Members of the control group packed only innocuous household items in a bag prior to proceeding to the simulated screening; they did not receive any information about the system or countermeasures training After packing the bag, participants proceeded to the simulated screening environment, purportedly to security screening prior to entry.

### Participants

In the first study, participants (N=172) were recruited from an introductory MIS course at a large southwestern university. Students participated in part to fulfill a course requirement. The course included students with diverse backgrounds from across the university. Although the ideal population to test would be people intent on committing a crime, such a population would be prohibitively difficult to find. Students can be considered to be an acceptable alternative for a proof-of-concept because the orienting and defensive theory and expected outcomes should be the same for a student population as for a criminal population. For instance, common drivers underlying the standard CIT have been found to be equally valid among general adult populations and antisocial or hyporesponsive criminals. Although students might not be the optimal test population (e.g., terrorists), the observations seen in this population likely have a useful level of external validity. Participants' average age was 23 years, 61.7% were Caucasian, 13.1% were Asian or Pacific Islander, 17.1% were Hispanic, and 6.3% were multi-racial. Participant gender was evenly split: 50% were female and 50% were male.

In the second study, participants were recruited from undergraduate and graduate business courses at a large southwestern university. While the ideal population would be individuals who regularly participate in illicit activities, such a population was not feasibly obtainable. Students were selected as a target population as students include individuals from a diverse set of ethnic and cultural backgrounds. Participants (N=175) consented to participate and subsequently completed the second study. The mean age of participants was 21.86, with a median age of 21, a minimum age of 18, and a maximum age of 36. The sample included individuals with a diverse range of ethnic backgrounds, including: 33% Asian, 3% Black, 1% Hawaiian/Islander, 46% White, 13% Hispanic, 3% Middle Eastern, and 1% other. Males accounted for 59% of the total participants; 41% were female. Over half (126) of the participants spoke English as a second language. Of the initial 175 participants, 18 were disqualified because they either failed two manipulation check questions or they did not follow instructions (e.g., failed to construct the device). Participants received course credit for participation, as well as $20 if they successfully made it through the checkpoint and completed their task without arousing suspicion. This was a mild deception to encourage personal investment; in reality no participants were required to complete their task beyond the screening.

### Experimental Task

In the first study, after completing a short questionnaire (for demographic data), participants received written instructions telling them to pack a bag and deliver it to an office in a secure portion of the building. To enter the secure portion of the building, the participants had to pass through a security screening process. Participants packed the bag with items provided (e.g., clothes, pad of paper, book, and other innocuous items). The instructions briefly mentioned that if the bag was judged suspicious, a secondary screening process would be necessary, which in the case of innocent participants would take additional time—thereby preventing them from delivering the bag in a timely manner.

Guilty participants were reminded that a more in-depth screening can expose their deception and prevent them from delivering the bag. This reminder proved sufficient motivation to successfully increase arousal, as guilty participants' self-reports and open-ended feedback given at the end of the first study showed increased arousal compared to their innocent counterparts.

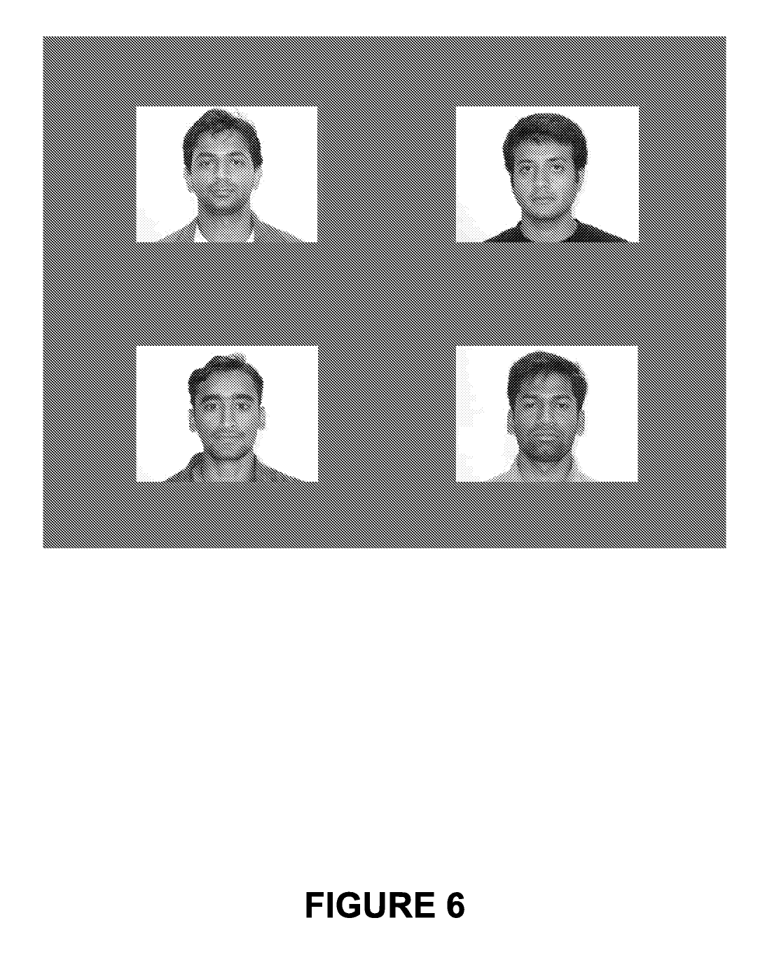

Participants in the guilty condition had two additional instructions. First, they received instructions on how to construct a mock explosive device (pre-assembled parts were provided) and were told to pack it in their bag along with the other items. A photograph of an example mock IED device used in the first study is shown in FIG. 5. Second, participants were shown a photograph of the face of the person to whom they were to deliver the bag. After packing the bag, each participant came to the screening room where he or she was interviewed by the ASK system.

In the second study, participants were randomly assigned to one of the three countermeasures groups also constructed the improvised explosive device and packed it in a bag; however, they also completed training on how to use countermeasures to defeat the system. Because deception detection is based on detecting behavioral or psychophysiological responses, the employment of countermeasures to appear innocent is a threat to the validity of any deception detection system.

The countermeasures training entailed watching a video describing the sensors that would be used during the screening interview to identify deception, as well as methods that could be used to thwart the effectiveness of the sensors. Participants in the mental countermeasures group were instructed to count backwards by seven from a three-digit number. This tactic is designed to occupy the examinee's mental processes, rendering them cognitively “numb” to the examinee that is taking place, minimizing any behavioral or physiological differences in behavior that could occur. Participants in the physical countermeasures group were instructed to moderately but carefully bite their tongue when control items were presented during the interview, resulting in physiological responses on control items that could mimic the expected response of a guilty person to an item of interest. This should result in a false negative classification as the guilty participant should appear more concerned about control items than items of interest, and would thus appear innocent. The final countermeasures group was trained to use the mental and physical countermeasures described previously, as well as to directly control kinesic rigidity (not “freeze up”), control their voice pitch such that it is always the same, keep a neutral facial expression, and to look at all images naturally, not focusing on one item on the screen more than another.

In both the first and second studies, after packing the bag, participants proceeded to the simulated screening environment, purportedly to security screening prior to entry. In the second study, participants entered the screening environment, placed the packed bag on a counter, and stood in front of the automated screening system.

For both the first and second studies, the ASK system asked five questions coupled with stimuli sets. In the first study, the first four questions read as follows: “The following items are not allowed beyond this point. Are you carrying any of these items?” Of the four boxes containing foil items that were subsequently presented, only one contained a word that was designed to relate to concealed information (i.e., “Bombs,” “Explosives,” “Weapons”) though all boxes contained a word describing a name of a banned class of items. FIG. 4 illustrates an example stimuli set for this four-question group. In the second study, the question was shortened to ask “Are you carrying any of these items?” and images related to concealed information, rather than words, were displayed by the ASK system.

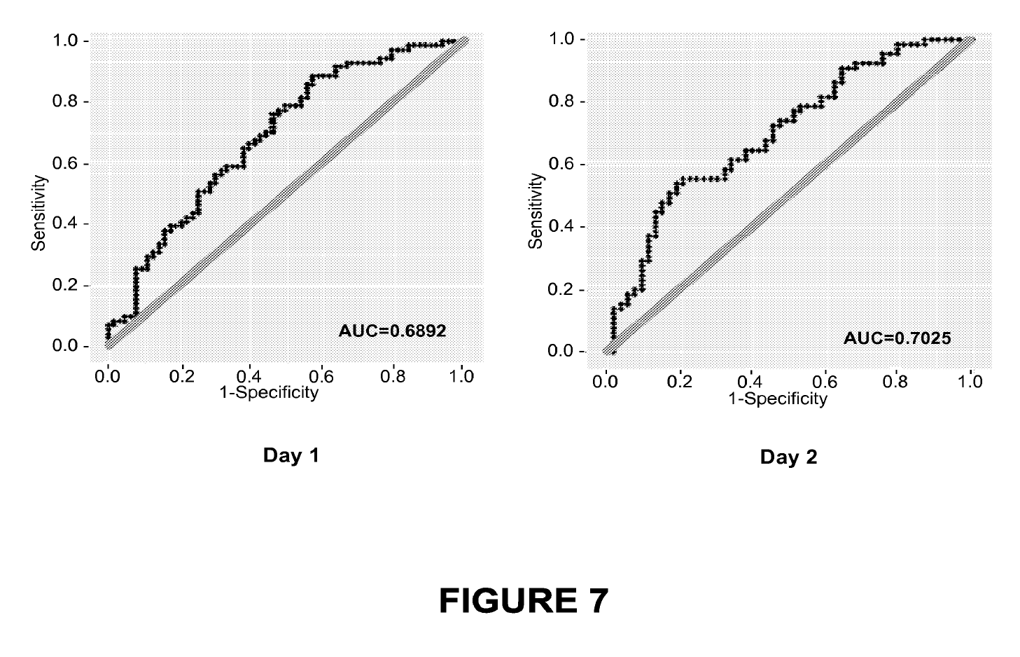

As an exploratory measure in the first study, an additional fifth question was presented by the ASK system computerized voice: “The following people are wanted by local authorities. Are you familiar with any of these people?” Immediately following the fifth question, the example four images of faces shown in FIG. 6 were displayed on the screen (see FIG. 6). Note that the format of FIG. 6 in format to the stimuli set displayed for the first four questions, such as the example stimuli set shown in FIG. 4. The example images shown in FIG. 6 were provided by Minear and Park.

Only one of the faces shown in FIG. 6 represented information that guilty participants would desire to conceal—that image represented the same person to whom they were directed to deliver the IED in the instructions that had preceded the screening process. To encourage a sense of realism, images of faces that were likely to remind participants of a stereotypical individual who might be involved in terrorist activity were chosen. Faces with similar features were chosen to ensure an inordinate amount of attention would not be drawn to a particular face simply because it possessed features that stood out as compared to the others.

### Example Measures

The eye-tracking system generated raw data in a Cartesian coordinate format. Two measurements were derived for each participant and stimuli set combination. The first was the initial direction of the first saccade after each question (dummy coded as “1” if toward a critical foil item). This calculation reflects a cognitive psychology measure for determining initial attention. The second measure was the percentage of time spent gazing at the safety point location during the time provided for a response. Similar measures of time spent gazing at a particular stimulus have been employed as surrogates for attention in human-computer interaction, marketing, and cognitive psychology. However, this context is somewhat unique in that increased dwell time at the center point of the screen is expected to reflect attention toward a topic represented elsewhere on the screen.

Kinesic data was captured using a Microsoft Kinect sensor, by capturing the 3-dimensional Cartesian coordinates of 20 distinct body points in real time at approximately 30 Hz. This raw data was used to calculate overall movement via the same procedure detailed in previous work on automated kinesic rigidity detection. This raw data was averaged by participant (N=157) and slide (N=20) for each body point. There was one case of lost data due to malfunction, leaving 62,780 data points.

Pupillometry was measured via an EyeTech™ TM3 eye tracking sensor. The TM3 captured pupil diameter at approximately 30 Hz. The mean pupil diameter for both eyes was calculated for each measurement and this data was summarized by participant and slide. Technical malfunctions occurred on 2.6% of slides, leaving 3,058 usable data points.

### Example ASK System Experiment Analysis and Results

In the first study, of the 172 participants, 36 were disqualified either because they did not follow instructions (23 cases) or because the system calibration process was unsuccessful (13 cases). Data from the remaining 136 participants (63 guilty, 73 innocent) were used in the analysis.

Manipulation check questions were included in the post-survey to determine 1) whether guilty participants possessed concealed information and 2) whether the concealed information was perceived as aversive by participants. The vast majority of participants (57 of 61, or 93%) in the guilty condition correctly reported that they had carried an illicit item through security screening. On the second day, 54 of 57 (95%) in the guilty condition correctly reported their attempt to carry an illicit item through screening. All participants in the innocent condition correctly reported on both days that they did not carry an illicit item. For each stimulus word, all participants reported how much attention that stimulus captured on a 7-point scale to gauge the word's perceived signal value. On day 1, participants in the guilty condition rated the signal value of critical items higher than non-critical items (F_{(1,120)}=107.9, η²=0.47, p<0.001), whereas innocent participants reported no significant difference (F_{1,114)}=2.8, η²=0.02, p=0.095). Day 2 showed similar results, with guilty participants reporting higher signal value on critical items (F_{(1,112)}=37.7, η²=0.25, p<0.001) and innocent participants showing no such effect (F_{(1,132)}=2.1, η²=0.02, p=0.15).

Table 5 displays self-reported mean (M) and standard deviation (SD) signal value statistics.

TABLE 5
Statistics of Self-Reported Signal Value of Foil Items

| | Day 1 | Day 2 | Day 1 | Day 2 |
|---|---|---|---|---|
| Items | Guilty | Guilty | Innocent | Innocent |
| Critical Foil | M = 5.46, | M = 4.68, | M = 4.26, | M = 3.96, |
| Items | SD = 1.42 | SD = 1.87 | SD = 2.32 | SD = 2.26 |
| Non-critical | M = 2.69, | M = 2.71, | M = 3.70, | M = 3.45, |
| Items | SD = 1.52 | SD = 1.55 | SD = 1.65 | SD = 1.70 |

### Orienting and Defensive Responses

A multilevel regression model was specified (N=1020) using mean time gazing at the Safety point (center of the screen) as the response variable (see H1 and H3). The participant (N=136) was treated as a random factor, while the experiment condition, stimuli set type (baseline or charged), and participation day were treated as fixed effects. Question order and target item position on the screen were included as covariates. When a stimuli set was charged (i.e., contained a critical item), only participants in the guilty condition spent significantly more time (4.5%) gazing at the safety point (t₍₁₀₁₃₎=3.06, p<0.01), as predicted by H1. The strength of this effect significantly increased to 8% on day 2, again only among participants in the guilty condition (t₍₁₀₁₃₎=2.70, p<0.01). The finding of significant effects on both days provides support for H3. Location of the target item and time were not significant factors. When the stimuli set containing the word “Bombs” was the first set presented, the strength of the effect was more pronounced than when the first presented set included “Explosives” or “Weapons.” Table 6 summarizes the multilevel regression results.

TABLE 6
Oculomotor Avoidance Behavior (Gazing at the Center of the Screen)
as the Response Variable for the First Four Stimuli Sets

| | | β Standard |
|---|---|---|
| Fixed Effects | β | Error |
| Intercept | 0.110*** | 0.014 |
| Concealed Information (i.e., Guilty) | 0.005 (n.s.) | 0.019 |
| Participation Day | 0.000 (n.s.) | 0.009 |
| Presence of Target Item | 0.016 (n.s.) | 0.010 |
| Concealed Information: Participation Day | 0.035** | 0.013 |
| Concealed Information: Presence of Target Item | 0.045** | 0.015 |
| Foil Presentation Order 2 | −0.021* | 0.010 |
| Foil Presentation Order 3 | −0.022* | 0.011 |

Note:
model fit by maximum likelihood.
***p < .001;
**p < .01;
*p < .05;
(n.s.) not significant

To test model fit, the model was compared to an unconditional model that omitted any fixed effects, using deviance-based hypothesis tests. The fit of the current model was significantly better than that of the unconditional model: χ²(1, N=1020)=64.69, p<0.001.

An overall logistic multilevel regression model revealed no main effect of condition on the direction of the initial saccade. However, a non-significant but suggestive interaction effect of condition and participation day was noted (z(765)=1.79, p=0.07). Separate analyses for each day revealed that for participants with guilty knowledge, the initial saccade was biased toward the critical item during the second day of screening (z(360)=2.34, Nagelkerke R2=0.14, p=0.02) but not during the first day (z(404)=−0.04, Nagelkerke R2=0.12, p=0.88). These findings provide support for H2, but not H4.

The exploratory stimuli set involving faces was analyzed separately from the word-based stimuli sets. Multilevel regression models for the faces question were specified similarly to those used for questions involving word stimuli. Concealed information was associated with a 6% increase in the amount of time gazing at the center of the screen (t (249)=3.00, p<0.01). Participation day had no main or interaction effects. Condition had no significant effects on the initial saccade for the faces set.

In summary, the results indicate that guilty knowledge significantly affected the tendency to look toward the critical item in a foil and the tendency to avoid looking at any foil stimuli after initially detecting them. A prediction that guilty knowledge causes visual attention to orient toward a critical item in a CIT foil was partially supported, with significant results occurring on day 2 but not on day 1. The prediction that the orienting response would diminish over time was not supported. The prediction that defensive behavior would encourage visual attentiveness toward the safety point was supported. Finally, the prediction that the defensive behavior effect would remain constant over time was supported.

### Oculomotor Defensive Behavior Classification Accuracy

The goal of the current work was not to develop a deployable system with proven performance for field use but to lay a foundation for a new class of systems (i.e., ASCSS) and provide initial support for their potential. However, an accuracy analysis in this initial investigation is useful for establishing that the initial system has potential value.

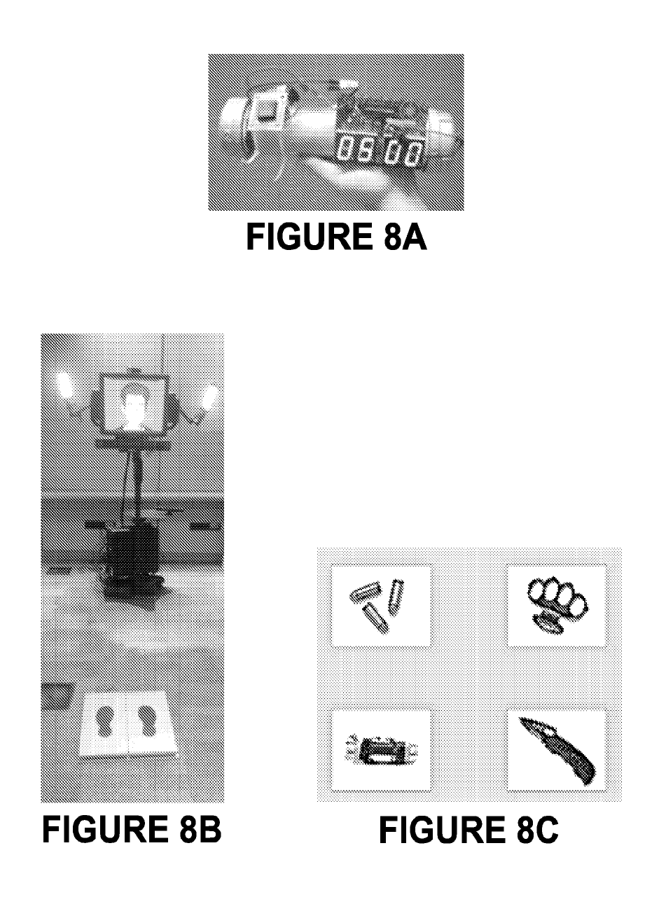

The problem of trying to detect concealed information in security screening can be conceptualized as a signal detection problem. Because oculomotor defensive behavior showed the strongest results, a receiver operating characteristic (ROC) analysis was performed on the data for each day, as shown in FIG. 7. Condition was positioned as the response variable, with gaze patterns positioned as the predictor variables. In a field setting, the acceptable cutoff rate for a risk score can vary based on estimated baseline rates, technological nuances, and operational considerations. ROC analyses are particularly helpful in estimating the accuracy rates that can be expected for any chosen risk threshold. For day 1, the oculomotor defense patterns produced an area under the curve (AUC) of 0.69; an AUC of 0.70 was produced from day 2 data. These can be interpreted as rough estimates of overall performance rates. Even at this early stage, these estimates exceed unaided human performance.

The purpose of this study was to propose and investigate ASCSS, a class of systems for autonomous scientifically controlled screening. Prior IS research on credibility assessment systems has focused mainly on between-group effects in unstructured interactions or open-ended responses, and practitioners rely on labor-intensive procedures and invasive sensors that introduce interviewer effects and limit application. The ASCSS design framework couples control for potentially confounding variables such as baseline interpersonal variations and interviewer effects with autonomous processing and noninvasive sensing. The ASK system, as a first instantiation of ASCSS, used visual stimuli sets and leveraged eye tracking as a noninvasive means of measuring orienting and defensive responses.

### Summary of Example Results

Alternative indicators of concealed information were successfully implemented—allowing for noninvasive and non-contact measurement. The results supported predictions that oculomotor defensive behavior would be revealed in participants who possessed purposely hidden information. As predicted, participants carrying a mock IED tended to avoid gazing at foil items—choosing to spend more time gazing at the center of the screen (i.e., the “safety point”) at which the expected visual stimulus was unrelated to the test. This effect remained constant even on the second day of participation when participants were familiar with the ASK system. The orienting response was measured by measuring eye-movement patterns (i.e., oculometrics). Participants who carried the mock IED were more likely to orient their initial visual attention toward the target item presented by the ASK system. However, this effect was seen only on the second day of participation. Whether this effect remains constant or decreases with multiple exposures is unclear. Table 7 summarizes the empirical results of the proposed oculomotor cues to concealed information.

TABLE 7
Empirical Outcomes of the ASK System Evaluation

| Measurement | | |
|---|---|---|
| Type | Expected Oculomotor Outcome | Result |
| Orienting | H1. Concealed information will increase the | Partially |
| response | likelihood that an initial saccade will be | Supported |
| | directed toward the target item in a collection | |
| | of simultaneously presented CIT foil items. | |
| Orienting | H2. Over time, stimuli representing concealed | Not |
| response | information will be likely to attract the initial | Supported |
| | saccade. | |
| Defensive | H3. Concealed information will cause | Supported |
| response | increased stimuli avoidance when the target | |
| | item is present. | |

### Design Principles

Driving the system design was the proposition that a scientifically controlled screening system like the ASK system can be automated and extended to non-traditional domains to discover valuable concealed information. Although further research is needed to refine the ASK system design, the initial results are promising. The ASK system operated automatically, with little or no need for manual intervention, and used a structured framework that generates a strong person-specific baseline to detect concealed information about IEDs at a rate greater than chance and unaided human judgment. In practice, additional sets with alternative target stimuli or questions strategically designed to address more than one topic at the same time would be required to identify potential concealment of other banned items or intent in a security screening context.

Tables 8 and 9 summarize the performance of the ASK system regarding the functional, performance, and process design guidelines.

TABLE 8
Functional and Performance Evaluation Results for the ASK System
Implementation

| # | ASK Performance |
|---|---|
| 1 | ASK operated autonomously using a scripted approach and ocular |
| | recognition. Manual intervention was required only for eye tracking |
| | sensor errors (<8% of cases). |
| 2 | No sensors required attachment. The eye-tracking system required |
| | minimal calibration, performed automatically in 10-15 seconds. |
| 3 | The selected sensor was operationally effective with approximately |
| | 92% of human examinees. |
| 4 | All examinees received exactly the same interview and gave exactly |
| | the same responses, virtually eliminating interviewing style, |
| | interviewer demeanor, response, and question-type effects. |
| 5 | The entire screening process required only two minutes from start |
| | to finish. |
| 6 | An ROC classification algorithm used the oculometric data to |
| | produce risk scores. |
| 7 | The ASK system as an initial prototype outperformed unaided |
| | human judgment. |

TABLE 9
ASK System Interview Protocol and Measurement Evaluation

| # | Ask Performance |
|---|---|
| 1 | Questions directly addressed the critical security screening goal |
| | of detection of banned items. |
| 2 | Words representing banned items were displayed in sets of four. |
| 3 | Only one word in each set represented an IED. Other words |
| | represented distinct banned items that were verifiably not present. |
| 4 | The ASK system collected oculometric data automatically. Those |
| | data were processed to identify visual orienting of attention and |
| | defensive gaze patterns for each question set. |
| 5 | Interpersonal differences were accounted for using multilevel |
| | regression analysis. |

When it comes to systems for structured credibility interviewing, the technology, protocols, and measurements commonly used today exhibit few substantial differences from that used 50 years ago. Because of a core competence in the synthesis of technologies, processes, and theories, the information systems discipline is uniquely positioned to generate useful knowledge that can have a strong impact on credibility assessment and human integrity management problems worldwide.

The positive results of this ASCSS investigation indicate potential for widespread credibility assessment. Researchers and practitioners can use ASCSS to assess virtually anything that is purposely hidden. For instance, ASCSS interviews can determine which employees are likely to be leaking sensitive company information. In locations where privacy laws allow, businesses can use ASCSS to improve internal security or help prevent insecure behavior. ASCSS interviews can also become part of periodic security policy compliance evaluations: Where an employee is not willing to openly admit negligence or mistakes regarding secure behavior, ASCSS can help discreetly determine which policies are likely to be a concern for particular individuals or groups of individuals. Similar automated adaptations of ASCSS can be used in pre-employment screening or to uncover insider threats such as classified information being sold or internal fraud. Other example contexts include consumer marketing research, corporate audits, employee performance reviews, and corporate negotiations. The noninvasive, low-cost, and rapid nature of ASCSS should be a welcome contrast to traditional extended interviews that use SCR and cardiorespiratory monitors, as well as the more recent CIT-based techniques using fMRI—all of which can be prohibitively invasive and expensive for widespread use in practice.

In instantiating and evaluating the ASCSS concept, this study also contributes unique oculomotor indicators of concealed information to the body of research. Oculometrics have been used in various research paradigms for more than a century and have recently been applied in IS and deception detection research. However, this study is among the first to use eye movement and the first to use oculomotor defensive behavior as an indicator of purposely concealed information. Oculomotor orienting has established traditions in cognitive psychology research for investigating perception and memory and in marketing and HCI research for investigating interest and intuitiveness. This study is unique in that orienting is examined within a high-stakes context where individuals are motivated to conceal their knowledge, thereby stimulating defensive eye movement.

### Kinesic Rigidity

In the second study, kinesic rigidity was detected among all groups who were smuggling the IED. When the target item was present on the screen, Guilty (b=−0.076, p=0.006), physical countermeasures (b=−0.203, p=0.000), mental countermeasures (b=−0.097, p=0.001), and all countermeasures (b=−0.063, p=0.024) groups exhibited kinesic rigidity. Those performing only physical countermeasures showed the greatest amount of kinesic rigidity, large enough to create a significant net positive main effect in that condition because of the standardized scoring method.

### Pupil Diameter

In the second study, when the target item (IED image) was displayed on the screen while a human examinee responded to a question, pupil dilation was significantly larger for participants in the Guilty (b=0.611, p<0.001), mental countermeasures (b=0.835, p<0.001), physical countermeasures (b=0.639, p<0.001) and all countermeasures (b=1.022, p<0.001) group, which showed the largest effect. There were relatively narrow pupil main effects of each condition resulting from the pronounced pupil dilation that occurred when target items were present, since all items were standardized within foil.

### Kinesic Rigidity and Pupil Dilation Results

Regarding kinesic rigidity and pupil dilation, overall body movement and pupil dilation had been investigated in CIT interviews previously, and the results replicated prior work showing kinesic rigidity and dilated pupils during presentation of target items. Traditional countermeasures were not effective at countering these behavioral and physiological responses, in agreement with kinesic rigidity predictions but contrary to expectations for pupil dilation. Pupil dilation was the strongest effect among those investigated and appeared to be the most resilient to countermeasures. The pupil dilation resulting from the orienting response was strong, and there was no decrease in this effect when mental distraction or pain was used.

Attempting many countermeasures at the same time proved difficult. When individuals tried to control many things at once, the pupil dilation effect was strongest. Physical countermeasures produced the strongest levels of kinesic rigidity. These findings may be used to detect specific countermeasures.

Key design science knowledge contributions include conceptualizations of a novel problem domain and solution. Herein is described the application domain for ASCSS and presented informed design guidelines for ASCSS systems, and is presented a prototypical instantiation and a large-scale evaluation of the prototype in order to better understand and refine the ASCSS concept.

### Countermeasures and Automated Screening Systems

Frequently, when new tests or protocols are developed, they are initially claimed to be resistant to countermeasures. In many cases, the matter is not that countermeasures will not work, but simply that the same countermeasures previously employed in other deception detection tests do not apply in the new test. The approach to countermeasures discussed herein took a similar approach, but with an added systems-inspired proposition—triangulating on deception through measurement of multiple behavioral and psychophysiological anomalies simultaneously. The findings suggest that this approach may be effective in some areas (e.g., pupil dilation and body movement), but not others (e.g., vocal pitch). The results are promising enough to justify additional research investigating countermeasure combinations at a more granular level.

The herein-disclosed technologies that can identify deception and concealed information rapidly and without contact have the potential to be used in a variety of interviewing and screening contexts, changing how integrity and security are managed. As with the polygraph, these new credibility assessment systems will encounter some individuals who will attempt to mitigate their effectiveness through the use of countermeasures.

### Deception Indicators in a Controlled Human Screening System

Whereas traditional deception detection technologies measure one or two distinct indicators, next-generation screening systems may show increased resilience to countermeasures if they capture multiple types of signals from multiple underlying behavioral or psychophysiological processes. To the extent individuals are limited in the number of activities to which they can be simultaneously attentive, they should be less able to counter a deception detection system that tracks and measures multiple heterogeneous indicators of deception.

In a third study, six indicators of deception were measured with the intent of capturing a broad range of indicator types. Three deception indicators—pupil dilation, kinesic rigidity, and gaze aversion—have previously been shown to be reliable indicators of deception within the context of a highly controlled screening system. Pupil dilation and kinesic rigidity are discussed in more detail in the sections “Facial Movement, Pupil Dilation, and Kinesic Rigidity Measures” and “Countermeasures” above.

Three of the deception indicators are exploratory in that they have not been previously examined in this specific context. These include vocal pitch, proximity, and frowning. Each of these deception indicators theoretically captures some distinct correlate of deception, and therefore may be differentially affected by physical and mental countermeasures. These deception indicators can be observed in a human examinee without contact using one or more sensors; e.g., cameras, eye-tracking devices, proximity sensors, microphones. In some cases, these sensors can detect small differences in motion and/or movements of the examinee that are normally, and perhaps wholly, imperceptible by unaided human beings.

The third study extends system design for the CIT method of presenting several baseline stimuli (representations that are not relevant to the illicit activity in question) together with relevant stimuli. Thus, similar results to previous work during presentation of relevant stimuli compared to baseline stimuli are anticipated. Table 10 summarizes hypotheses tested by the third study.

TABLE 10
Hypotheses Tested During Third Study Regarding Deception Indicators

| Hypothesis | Stimuli | Description |
|---|---|---|
| H1 | Pupil | Deceptive individuals will have a larger |
| | Dilation | pupillary response to target items than to |
| | | non-target items. |
| H2 | Pupil | Deceptive individuals using mental |
| | Dilation | countermeasures will exhibit increased pupil |
| | | dilation during responses to target items. |
| H3 | Pupil | Physical countermeasures will reduce the pupil |
| | Dilation | dilation differential between target and non- |
| | | target items. |
| H4 | Kinesic | Deceptive individuals will exhibit less overall |
| | Rigidity | movement when viewing and responding to a |
| | | target item. |
| H5 | Kinesic | Deceptive individuals employing mental or |
| | Rigidity | physical countermeasures will exhibit less |
| | | overall movement when viewing and |
| | | responding to a target item. |
| H6 | Gaze | Deceptive individuals will show increased gaze |
| | Aversion | time at the screen center when presented with a |
| | | target item. |
| H7 | Gaze | Deceptive individuals employing mental or |
| | Aversion | physical countermeasures will exhibit greater |
| | | gaze duration at the screen center when viewing |
| | | and responding to a target item. |
| H8 | Vocal | Deceptive individuals will exhibit greater vocal |
| | Pitch | pitch when responding to a target item. |
| H9 | Proximity | Deceptive individuals will move closer to a |
| | | target item. |
| H10 | Frowning | Deceptive individuals will increase frowning |
| | | when presented with a target item. |

Gaze Aversion

Research supports the hypothesis that when presented with several equidistant stimuli on a single screen, individuals concealing guilt have a tendency to spend more time gazing away from all stimuli by spending more time looking at the center of the screen. This effect happens if one of the stimuli is highly associated with the guilt being concealed.

This gazing tendency may stem from an autonomic avoidance response, or it may be an overt defensive behavior designed to help avoid suspicion. Should this tendency stem from overt defensive behavior, traditional countermeasures designed to mentally distract or corrupt physiological measurement readings should not naturally translate into controlling visual gaze.

Vocal Pitch

Whereas the above-named correlates of deception stem from autonomic psychophysiological processes and overt defensive behavior, vocal pitch is thought to correlate more with emotional stress. To speak, the diaphragm pushes air through vocal folds in the larynx. The frequency of the air pressure changes affected by vibration of the vocal folds is perceived as the vocal pitch. The vocal fold vibrations are facilitated by muscles about the larynx in the vocal tract. Just like other muscles in the body, the larynx muscles exhibit tension when an individual experiences stress or arousal. Tension around the larynx causes vocal folds to increase the frequency of vibration, thereby increasing vocal pitch.

Increases in mean and range in vocal pitch have been predictive of deceptive speech and heightened emotions and arousal. Because vocal pitch provides primarily emotional arousal-based information, it has not been analyzed in a controlled interview such as the CIT, as these traditionally rely on psychophysiological measurements. However, it is likely that emotional arousal may be present in a controlled interview even though it has not traditionally been measured.

Mental countermeasures may be effective against vocal pitch to the extent they are able to distract enough to diminish emotional reaction. However, physical countermeasures such as stepping on a tack or even biting one's tongue may not cause tension in the larynx muscles, so they may not be effective.

Proximity

Interpersonal proximity—the physical distance between two social actors—has been hypothesized to increase with deception in interpersonal communication. This view assumes proximity is a type of non-verbal immediacy, which is the degree to which a communication is direct, relevant, clear, and personal. However, in an automated screening interaction where allowed responses are highly restricted, immediacy is not likely to vary. In one relevant study that used a structured interaction and a virtual screening agent, proximity significantly decreased with deceivers. It is possible that proximity decreases slightly as a function of the orienting reflex: a person may reflexively move slightly closer to a target as they allocate more attention toward it.

Frowning

When it comes to facial expressions, “leakage” of emotional indicators is the dominant explanation for deception indicators. When the act of lying and/or perceptions of guilt generate negative affect, those emotions have a natural tendency to show up in the face. Some evidence supports the notion that controlling facial expressions can be a difficult venture, though it may be easier to hide less intense emotions. However, little or no research has investigated leaked emotion in facial expressions in a controlled, automated screening interview, where there is no natural, free-flowing, dynamic conversation. In such a setting, deceivers' emotions should remain constant except when presented with target stimuli, when increased negative affect such as guilt or fear could lead to a more negative expression.

It is especially important to determine whether individuals can be successful when they attempt to counter many factors at once. Thus, in addition to testing the hypotheses listed in Table 10, the third study also investigates whether countermeasures are less effective when multiple countermeasures are employed simultaneously.

Research Approach for the Third Study

The third study both evaluated overall system performance and tested hypothesized outcomes. An experiment was designed to evaluate the ability of deceivers to successfully bypass an automated screening system through the use of countermeasures. The experimental task was patterned after a number of experiments designed to test the ability of non-invasive sensors to identify deception and concealed information. The experiment included five conditions—four guilty and one control. Three of the four guilty conditions included learning certain countermeasures. Measures were repeated within-subjects and within-questions for a total of 20 measurements (captured during responses to 20 questions) per individual, per indicator.

Participants of the Third Study

Participants were recruited from undergraduate and graduate business courses at a large American university. Human subject review approval was obtained and all human subject procedures were followed. While the ideal population would be individuals who regularly participate in illicit activities, such a population was not feasibly obtainable. Students were therefore selected as a target population due to (1) the cultural and ethnic diversity of student populations and (2) the empirically-supported similarities between physiological response patterns exhibited by examinees in both field and laboratory controlled interviews. Participants' (N=175) mean age was 21.86, with a median age of 21, a minimum age of 18, and a maximum age of 36. The sample included individuals with a diverse range of ethnic backgrounds, including: 33% Asian, 3% Black, 1% Hawaiian/Islander, 46% White, 13% Hispanic, 3% Middle Eastern, and 1% unidentified. Females accounted for 41% of the total participants. Over half (126) of participants spoke English as a second language. Of the initial 175 participants, 18 were disqualified because they either failed two manipulation check questions or they did not follow instructions (e.g., failed to construct the device). Participants received course credit for participation, as well as $20 if they successfully made it through the checkpoint and completed their task without arousing suspicion. This was a mild deception to encourage personal investment; in reality no participants were required to complete their task once they passed beyond the screening.

Experimental Task for the Third Study

Each participant was required to provide written consent prior to beginning the third study. After providing consent, each individual was randomly assigned to a group having one of the following five conditions: innocent (as a control group), a guilty with no countermeasures, guilty with mental countermeasures, guilty with physical countermeasures, and guilty with all (mental, physical, and additional) countermeasures. The number of participants assigned to each group was roughly equal.

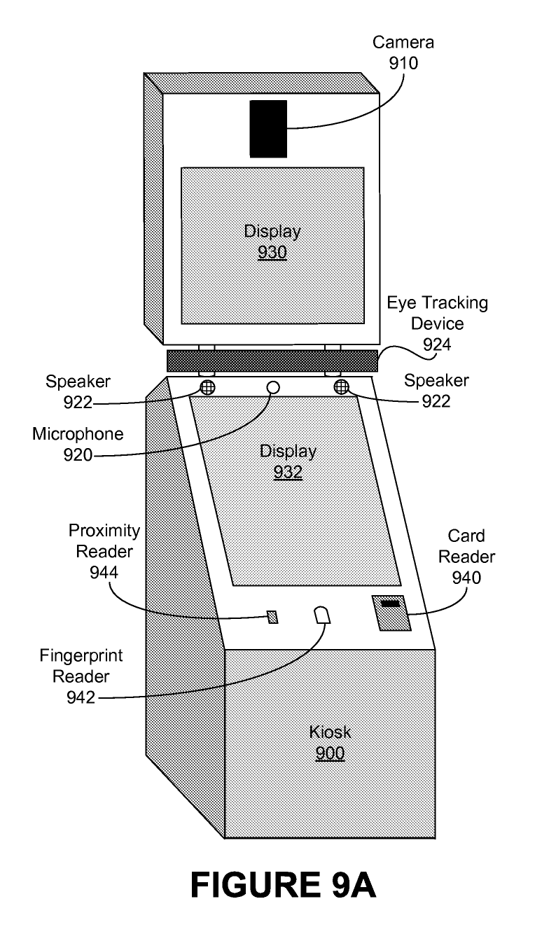

Each participant packed a bag and proceeded to a simulated screening environment, purportedly for security screening prior to entry. Each member of the innocent/control group packed only innocuous household items in their bag prior to proceeding to the simulated screening; they did not receive any information about the system or countermeasures training Participants in each of four guilty conditions groups were tasked with constructing an improvised explosive device, concealing it in a bag with standard travel items, and delivering the bag to a person located in a secure portion of a building. FIG. 8A is a photo of the improvised explosive device. Participants in the three countermeasures groups also completed training on how to use their assigned countermeasure(s) to defeat the system.

Countermeasures training entailed watching a video describing the sensors that would be used during the screening interview to identify deception, as well as methods that could be used to thwart the effectiveness of the sensors. Participants in the mental countermeasures group were instructed to count backwards by seven from a three-digit number. This tactic is designed to occupy the examinee's mental processes, thereby cognitively blocking out most of the screening interview that is taking place, minimizing any behavioral or physiological differences in behavior that could occur. Participants in the physical countermeasures group were instructed to moderately but carefully bite their tongue when baseline stimuli were presented during the interview, resulting in physiological responses on baseline stimuli that could mimic the expected response of a guilty person to an item of interest. This should result in a false negative classification as the guilty participant should appear as much or more concerned about control items than items of interest, and would thus appear innocent. The “all” countermeasures group was trained to use the mental and physical countermeasures described previously, as well as to directly control kinesic rigidity (not “freeze up”), control their voice pitch such that it is always the same, keep a neutral facial expression, and look at all images naturally—not focus more on one stimulus on the screen than another.

Upon entry at the simulated screening environment, all participants placed the packed bag on a counter and stood in front of the automated screening system shown in FIG. 8B for examination via a screening interview. The system was designed to identify presence of an examinee and begin the interview autonomously. The first step of the interview entailed calibration of the examinee to the eye tracking device. During this process, the examinee fixates their eye gaze on nine different locations on the screen, resulting in a more accurate and consistent oculometric data collection.

After calibration, the interview commenced, which entailed displaying five slides containing images of banned items to the examinee. Each slide contained four small images of banned items (example slide shown in FIG. 8C), and an embodied conversational agent (ECA) asked the examinee “Are you carrying any of these items?” immediately prior to the display of each screen. The examinee was given five seconds to respond before the question repeated and the next slide appeared. Participants were asked to vocally respond either “yes” or “no.”

One of the five slides contained the target item, which was an image that resembled an explosive device. Participants in one of the four guilty conditions groups had to verbally lie in response to this question to appear truthful. The five-slide set was repeated four times, with the slide order and the spatial location of the images changed each time. In total, each examinee responded to 20 questions, four of which required an outright lie for a guilty condition group member. After the interview, each examinee completed a brief survey designed to check experimental manipulations and to collect information on their experience during the interview.

Experimental Measures for the Third Study

Kinesic data was captured using a Microsoft Kinect sensor, which captured three-dimensional Cartesian coordinates of 20 distinct body points in real time at approximately 30 Hz. This raw data was used to calculate overall movement via the same procedure detailed in previous work on automated rigidity detection. This raw data was averaged by participant (N=157) and response (N=20) for each body point. Proximity was measured using the same data and process, except the raw data used for input was distance from the sensor for each major body point.

Pupil diameter was measured via an EyeTech™ VT2 eye tracking sensor. The VT2 captured pupil diameter at approximately 30 Hz. The mean pupil diameter for both eyes was calculated for each measurement and the data was summarized by participant and response. Center gaze duration ratio was calculated using Cartesian coordinate data collected by the VT2. Percentage of time viewing the center of the screen was likewise calculated for each slide-participant combination.

Raw vocal data was captured at 48 kHz using an array microphone. For each response, the maximum, mean, and standard deviation of vocal pitch from the beginning to the end of an utterance of a “no” response were extracted from the raw vocal data.

Frowning was measured by analyzing video captured at 15 fps by a standard high-definition web camera. Frown data was generated from the videos using the Computer Expression Recognition Toolbox (CERT). CERT generates the level of smile (or frown) for each video frame using an algorithm trained on a database of diverse images of faces.

For each sensor and indicator, there were some cases of data loss due to misconfiguration, difficulty with calibration, or low-fidelity data capture. For instance, technical malfunctions with the raw vocal data sensor occurred on 5.6% of the slides, leaving 2,994 usable data points for that indicator. The number of usable data points for each indicator is listed as “N” in Table 11. To control for effects stemming from highly variable interpersonal differences such as wide variance in nervousness, stillness, eye size, and vocal range, the data points from each of these indicators were standardized using within-subject z-scores, meaning each subject's observations were representative of a personal baseline as opposed to a population baseline. All observations were also standardized within-foil, to take advantage of the question-specific baseline. In the case of body movement, movement was also standardized for each body point separately to account for natural differences in movement patterns between body points.

Analysis and Results of the Third Study

Because any of several measures of vocal pitch can be useful in a controlled screening context, vocal pitch variation underwent a preliminary analysis to explore three possible vocal veracity measures: mean pitch, pitch standard deviation, and max pitch. Then, separate multilevel regression analyses were performed for each veracity indicator. As with body movement and pupil dilation indicators, each of these variables were normalized within-subject and within-foil before being submitted to repeated measures ANOVA (Condition X Target Item). The interaction of Condition and Target Item was not significant for mean pitch (p=0.28) or pitch standard deviation (p=0.065). Max pitch, a measurement of high-end pitch, was significant for the Condition and Target interaction, F(4, 2989)=2.32, p=0.05.

For each target deception indicator of interest, a multilevel regression model was specified with the indicator as the dependent variable. The dependent variables are standardized scores representing standard deviations from an individual baseline. In each case, the independent variables included: a Target Item binary variable indicating whether the stimuli slide included the IED image, a Time variable with a value between 1 and 4 representing the temporal order of the four sets of stimuli slides, and a Condition variable (the four guilty conditions were dummy coded using the Innocent condition as the baseline). Interaction effects between the Condition and Target Item variables were included to test hypotheses. The results of the separate multilevel models are in Table 11.

TABLE 11
Multilevel Regression Results

| | | | Center | Max | | |
|---|---|---|---|---|---|---|
| | Pupil | Overall | Gaze | Vocal | | |
| | Diameter | Movement | Duration | Pitch | Proximity | Frown |
| Fixed Effects | β (S.E.) | β (S.E.) | β (S.E.) | β (S.E.) | β (S.E.) | β (S.E.) |
| (Intercept) | 0.090* | 0.007 | 0.046 | 0.077 | −0.032 | 0.010 |
| | (0.045) | (0.010) | (0.057) | (0.047) | (0.058) | (0.054) |
| Target Item | −0.074 | −0.026 | −0.239* | −0.247** | 0.160 | −0.049 |
| | (0.082) | (0.019) | (0.096) | (0.086) | (0.062) | (0.088) |
| Time | −0.008** | 0.000 | 0.000 | −0.003 | 0.000 | 0.000 |
| | (0.003) | (0.001) | (0.016) | (0.003) | (0.016) | (0.015) |
| Guilt | −0.123* | 0.015 | −0.037 | −0.061 | 0.058 | −0.086 |
| | (0.053) | (0.012) | (0.059) | (0.056) | (0.062) | (0.056) |
| Mental | −0.167** | 0.019 | −0.033 | −0.049 | 0.070 | 0.019 |
| Countermeasures | (0.054) | (0.013) | (0.064) | (0.057) | (0.063) | (0.058) |
| (MC) | | | | | | |
| Physical | −0.128* | 0.041** | −0.056 | −0.070 | 0.045 | 0.000 |
| Countermeasures | (0.054) | (0.013) | (0.061) | (0.057) | (0.063) | (0.056) |
| (PC) | | | | | | |
| All | −0.204*** | 0.013 | −0.048 | -0.040 | 0.022 | −0.001 |
| Countermeasures | (0.054) | (0.012) | (0.061) | (0.056) | (0.062) | (0.058) |
| (AC) | | | | | | |
| Guilt X Target | 0.611*** | −0.076** | 0.195 | 0.304* | −0.288* | 0.432*** |
| Item | (0.119) | (0.027) | (0.135) | (0.124) | (0.138) | (0.125) |
| MC X Target | 0.835*** | −0.097*** | 0.167 | 0.243 | −0.348* | −0.095 |
| Item | (0.121) | (0.028) | (0.148) | (0.130) | (0.140) | (0.013) |
| PC X Target | 0.639*** | −0.203*** | 0.289* | 0.348** | −0.226 | −0.002 |
| Item | (0.121) | (0.028) | (0.138) | (0.128) | (0.140) | (0.013) |
| AC X Target | 1.022*** | −0.063* | 0.250 | 0.198 | −0.112 | 0.002 |
| Item | (0.121) | (0.028) | (0.140) | (0.126) | (0.139) | (0.013) |
| N | 3058 | 62780 | 2434 | 2994 | 3139 | 2965 |

*p < .05; **p < .01; ***p < .001; models fit using maximum likelihood. Less overall movement = increased rigidity.

Each model was compared to an unconditional multilevel regression model that excluded fixed effects, and each explained significantly more variance than an unconditional model. The unconditional model partitions the variance across participants unconditioned by predictor variables. Comparing the unconditional models against each of the models enables testing whether the inclusion of the predictors significantly improves the fit of the model to the data before examining the fixed effects.

Confirmatory Indicators of the Third Study

When the target item (IED image) was displayed on the screen while a human examinee responded to a question, pupil dilation was significantly larger for participants in the guilty without countermeasures (b=0.611, p<0.001), mental countermeasures (b=0.835, p<0.001), physical countermeasures (b=0.639, p<0.001), and all countermeasures (b=1.022, p<0.001) groups, with the all countermeasures group showing the largest effect.

Kinesic rigidity was detected among all guilty (non-control) groups. When the target item was present on the screen, the guilty without countermeasures (b=−0.076, p=0.006), physical countermeasures (b=−0.203, p=0.000), mental countermeasures (b=−0.097, p=0.001), and all countermeasures (b=−0.063, p=0.024) groups exhibited rigidity. Those performing only physical countermeasures showed the greatest amount of rigidity.

Gazing at the center of the screen was not a significant indicator of deception in this study except for the physical countermeasures group (b=0.289, p<0.05), though the trend toward increased center gazing was consistent for all guilty condition groups.

Exploratory Indicators for the Third Study

Maximum vocal pitch was selected as the dependent variable for vocal pitch variation in the multilevel regression model detailed in Table 11. Both physical (b=0.348, p=0.007) and guilty without countermeasures (b=0.304, p=0.014) conditions demonstrated increases in max pitch when responding to target items. The mental countermeasures condition did not achieve significance, b=0.24, p=0.06. The condition using several countermeasures was not significantly different from the innocent condition, b=0.19, p=0.12. Proximity decreased among those in the guilty conditions, though significantly so only in the guilty with no countermeasures condition (b=−0.288, p<0.05) and the mental countermeasures condition (b=−0.348, p<0.05). Frowning significantly increased among participants in the guilty condition when a target stimulus was presented (b=0.432, p<0.001), but no difference was found for those in countermeasures conditions.

Predictive Capability Results for the Third Study

To examine robustness to countermeasures, the overall system was evaluated for its predictive capability compared to innocent responses when different countermeasure types were used. In many cases, it is straightforward to generate a post hoc prediction algorithm that achieves 100% accuracy on a given dataset. The generalizability of such algorithms, however, is questionable. To strengthen generalizability of predictive results in the current analysis, each prediction algorithm used a two-thirds/one-third training/testing split, and the training phase used ten-fold cross-validation. Missing values were imputed using a random forest approach. Several predictive algorithms were generated for each indicator or indicator group, including Naïve Bayes, Logistic Regression, Random Forest, and SVM.

A naïve ensemble algorithm equally weighted the output of each of these approaches, with ensemble results are reported in Table 12. This combination of actions is likely to result in conservative accuracy estimates. Although the best ensemble results produced an 86% accuracy, the best baseline performance came from using all indicators in a trained logistic regression model (90% overall, 90% sensitivity, 90% specificity).

TABLE 12
Prediction Capabilities of Indicators in a Controlled
Screening System

| | | | |
|---|---|---|---|
| Indicators | Accuracy | Sensitivity | Specificity |

Baseline (no countermeasures)

| | | | |
|---|---|---|---|
| All Indicators | 0.86 | 0.90 | 0.82 |
| Confirmatory Indicators | 0.86 | 1.00 | 0.64 |
| Exploratory Indicators | 0.67 | 0.40 | 0.90 |

Mental countermeasures

| | | | |
|---|---|---|---|
| All Indicators | 0.86 | 0.90 | 0.82 |
| Confirmatory Indicators | 0.81 | 0.90 | 0.73 |
| Exploratory Indicators | 0.52 | 0.10 | 0.91 |

Physical countermeasures

| | | | |
|---|---|---|---|
| All Indicators | 0.62 | 0.40 | 0.82 |
| Confirmatory Indicators | 0.62 | 0.40 | 0.82 |
| Exploratory Indicators | 0.57 | 0.50 | 0.64 |

All countermeasures at once

| | | | |
|---|---|---|---|
| All Indicators | 0.71 | 0.80 | 0.64 |
| Confirmatory Indicators | 0.80 | 0.70 | 0.91 |
| Exploratory Indicators | 0.33 | 0.20 | 0.45 |

Note:
Sensitivity = Detecting Guilt;
Specificity = Detecting Innocence

Summary of Results of the Third Study

Analysis of pupil dilation and general body movement replicated prior work showing kinesic rigidity (H4) and dilated pupils (H1) during presentation of target items in a controlled interview. Traditional countermeasures were not effective at countering these responses, supporting rigidity expectations (H5) and pupil dilation expectations with regard to mental countermeasures (H2), but contrary to expectations for pupil dilation (H3). Pupil dilation was the strongest effect among those investigated and appeared to be the most resilient to countermeasures. The pupil dilation resulting from the orienting response was pronounced, and there was no decrease in this effect when mental distraction or pain was used. Center-of-screen gaze appeared to increase among guilty participants during responses to relevant items, but contrary to prior research the increase was not statistically significant (H6, H7). The most likely explanation for this difference is the effect is simply not as pronounced as other indicators. Participants in the guilty with no countermeasures condition exhibited greater maximum vocal pitch (H8), closer proximity (H9), and increase frowning (H10), as hypothesized. A review of the results also suggests that mental countermeasures may be at least somewhat effective against vocal pitch, though physical countermeasures were not. Proximity significantly decreased, and unlike vocal pitch, proximity appears to be more robust to mental countermeasures than to physical countermeasures. Though increased frowning was significant in the baseline guilty condition, it was nonexistent in all of the countermeasures conditions, suggesting it may be easily overtly controlled.

Table 13 summarizes results from explicit hypotheses for the third study.

TABLE 13
Summary of Hypothesis Testing Results for the Third Study

| # | Hypothesis | Supported? |
|---|---|---|
| H1 | Deceptive individuals will have a larger pupillary | Yes |
| | response to target items than to non-target items. | |
| H2 | Deceptive individuals using mental countermeasures | Yes |
| | will exhibit increased pupil dilation during responses | |
| | to target items. | |
| H3 | Physical countermeasures will reduce the pupil dilation | No |
| | differential between target and non-target items. | |
| H4 | Deceptive individuals will exhibit less overall | Yes |
| | movement when viewing and responding to a target | |
| | item. | |
| H5 | Deceptive individuals employing mental or physical | Yes |
| | countermeasures will exhibit less overall movement | |
| | when viewing and responding to a target item. | |
| H6 | Deceptive individuals will show increased gaze time at | No |
| | the screen center when presented with a target item. | |
| H7 | Deceptive individuals employing mental or physical | No |
| | countermeasures will exhibit greater gaze duration at | |
| | the screen center when viewing and responding to a | |
| | target item. | |
| H8 | Deceptive individuals will exhibit greater vocal pitch | Yes |
| | when responding to a target item. | |
| H9 | Deceptive individuals will move closer to a target item. | Yes |
| H10 | Deceptive individuals will increase frowning when | Yes |
| | presented with a target item. | |

In general, the prediction capability of a controlled screening system appeared to be more robust when multiple indicators were used for prediction. Mental countermeasures can alter behavior and physiology in various ways, but these changes are not likely to effectively undermine the effectiveness of the system. Although mental countermeasures appeared to be not very effective at a general level, physical countermeasures manipulated behavior and physiology to an extent that system performance significantly decreased. Performance likewise decreased when multiple types of countermeasures were attempted simultaneously, although not to the same degree. It is possible that physical countermeasures were the key driver of the performance drop in this group as well.

The third study indicates that pupil dilation as a function of the orienting response appears to be strongest and most robust indicator. In fact, this indicator is strongest when many countermeasures are attempted simultaneously, suggesting it is very difficult to counter in this setting. Kinesic rigidity and gazing at the screen center are relatively weaker, though physical countermeasures strengthened these two indicators. These may prove valuable for detecting physical countermeasures, which is an apparent weakness of this type of system.

While rigidity and pupil dilation indicators were more robust to countermeasures, exploratory indicators of vocal pitch, proximity, and frowning were clearly affected, suggesting that they are more overtly controllable. Depth of frowning in particular appears to be easily controlled, even when attempting to control multiple behaviors simultaneously.

Example Kiosk with Embedded Species Agents

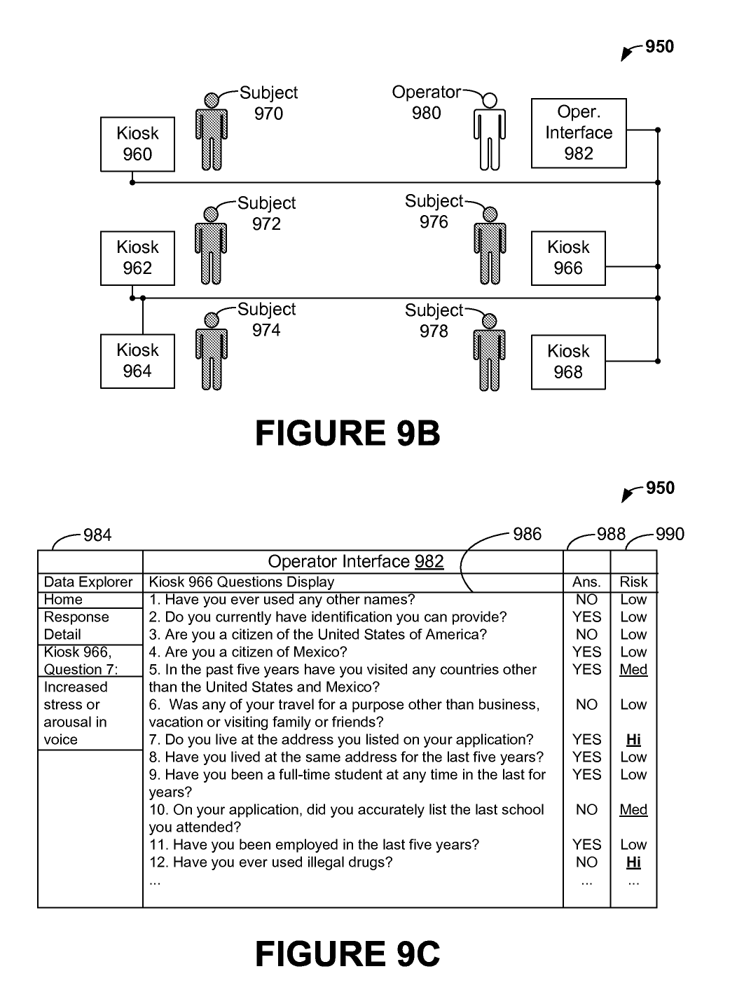

FIG. 9A shows an example kiosk 900. As shown in FIG. 9A, kiosk 900 can contain several sensors, such as a high-definition video camera 910, microphone 920, eye tracking device 924, displays 930, 932, card reader 940, fingerprint reader 942, and proximity reader 944. In some cases, at least display 932 is configured with a touch screen to enable touch-based input; e.g., as provided by a human subject using kiosk 900. In other cases, kiosk 900 can be configured with output devices, such as speakers 922 for audible outputs and displays 930, 932 for visual outputs. In some embodiments, kiosk 900 can be equipped with additional cameras, such as a near-infrared and/or infrared cameras, still cameras, and/or other types of cameras.

Kiosk 900 can be used to interview a human subject. For example, an “avatar” or image(s) representing an embedded conversational agent (ECA) can be displayed; e.g., using monitor 930, and ask questions of the human subject via speech (and perhaps other sounds) emitted using speakers 922. The ECA can include avatars having full physical representations, or just a part of the body such as a head and face. There are several reasons to use an embodied face over only sound and text when communicating and interacting with individuals. The face, especially the lower face, can be very useful conveying emotions visually, and so embodied agents can effectively communicate an intended emotion through animated facial expressions alone.

An ECA can utilize human interaction as a control component. Humans manifest a state of arousal through several physiological responses including pupil dilation, change in heart rate and blood pressure, change in blood flow, increase in body temperature, especially around the face and eyes, and changes in blink patterns. Sensors of kiosk 900 can capture both physiological and behavioral cues from the human counterparts. Physiological cues that may be diagnostic of emotional state, arousal, and cognitive effort include heart rate, blood pressure, respiration, pupil dilation, facial temperature, and blink patterns. Behavioral indicators include kinesics, proxemics, chronemics, vocalics, linguistics, eye movements, and message content.

The human subject can provide direct input using the touch screen of display 932 for touch-based inputs and/or microphone 920 for speech-based inputs. The human subject can also be observed using camera 910. Kiosk 900 can accept documentation related to the human subject; e.g., passports, identity cards, etc. For example, kiosk 900 can accept documentation via proximity reader 940; e.g., for reading Radio Frequency ID (RFID) provided documentation and/or card reader 944; e.g., for reading one-dimensional (bar code) and two-dimensional (QR code) encoded information, magnetic media encoding documentation, and/or alphanumeric documentation. Also, the human subject can provide kiosk 900 with fingerprint data, as needed, using fingerprint reader 942.

In other embodiments, kiosk 900 can be configured with more, different, and/or fewer sensors and/or output devices; e.g., kiosk 900 can be configured with a laser-Doppler vibrometer, different types of cameras, and/or eye tracking sensors. In some other embodiments, card reader 944 can include an electronic passport reader, such as the 3M AT-9000 electronic passport reader. The e-passport reader can read information from a document, such as a passport or visa, and/or capture an image of the document.

FIG. 9B illustrates an example environment 950 where five kiosks 960, 962, 964, 966, 968 are operating simultaneously to interview five subjects 970, 972, 974, 976, and 978. For example, environment 950 can be at a border crossing location or port of entry, where subjects 970-978 are being interviewed about their respective immigration and/or customs status.

Kiosks 960-968 are being operated by operator 980, who can observe questioning, review answers, and observe kiosk operation via operator interface 982 to kiosks 960-968. In question display includes questions asked by “Kiosk 966”. In some embodiments, operator interface 982 can permit operator 980 to switch between kiosks being reviewed; e.g., to switch from a current kiosk; e.g., “Kiosk 966” to another kiosk; e.g., kiosk 964. In still other embodiments, operator interface 982 can permit simultaneous review of multiple kiosks; e.g., operator interface 982 can provide multiple windows, where each window provides a display for a predetermined kiosk, such as the display for kiosk 966 shown in FIG. 9C.

Answer display 988 shows answers provided by subject 976 to questions 986 at kiosk 966. In some embodiments, such as shown in FIG. 9C, the answers can be “Yes” and “No” answers; while in other embodiments, other answers can be provided, such as, but not limited to, numerical answers, additional words beyond “Yes” and “No”, image files, video files, and sound files. Each answer in answer display 988 corresponds to a question in question display 986; e.g., question 1 in questions display 986 of “Have you ever used any other names?” is shown to be answered with a “NO” in answer display 988.

Risk assessment display 990 shows a risk assessment for each answer shown in answer display 988. For example, risk assessment display 990 shows a “Low” risk for the answer “NO” shown in answer display 988 to the “Have you ever used any other names?” question shown in question display 986. In the embodiment shown in FIG. 9C, a “Hi” for high risk, “Med” for medium risk, and “Low” risk scale is used to display risk, with each of the “Hi”, “Med”, and “Low” risks being displayed using different textual styles. FIG. 9C shows the “Hi” risk assessment using underlining and a bold font; e.g., Hi, the “Med” risk assessment using underlining; e.g., Med, and the “Low” risk assessment in using an unaccented or normal font; e.g., Low. In some embodiments, the risk assessment can be displayed using numerical values, colors, images/icons, and/or using other techniques. In particular embodiments, answer display 988 and risk assessment display 990 can be combined; e.g., a displayed answer can be shown using “stoplight colors”: a red color to indicate a high risk assessment, a yellow color to indicate a medium risk assessment, or a green color to indicate a low risk assessment. Other techniques for exploring data and displaying questions, answers, and risk assessments are possible as well.

### Example Computing Environment

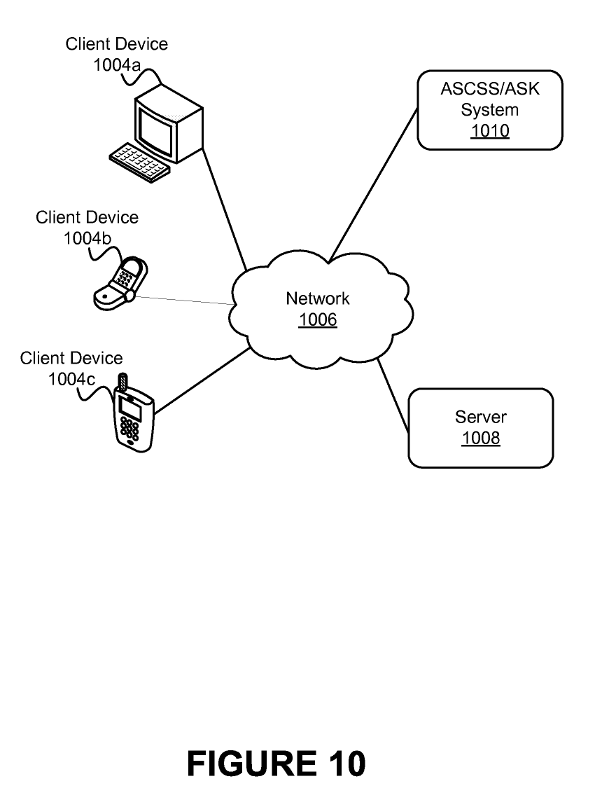

FIG. 10 is a block diagram of an example computing network. Some or all of the above-mentioned techniques disclosed herein, such as but not limited to techniques disclosed as part of and/or being performed by a kiosk, an ASCSS, and/or ASK system can be part of and/or performed by a computing device. For example, FIG. 10 shows ASCSS/ASK system 1010 configured to communicate, via network 1006, with client devices 1004a, 1004b, and 1004c and server 1008.

Network 1006 may correspond to a LAN, a wide area network (WAN), a corporate intranet, the public Internet, or any other type of network configured to provide a communications path between networked computing devices. Network 1006 may also correspond to a combination of one or more LANs, WANs, corporate intranets, and/or the public Internet.

Server 1008 can be configured to perform one or more services, as requested by programmable devices 1004a, 1004b, and/or 1004c. For example, server 1008 can provide content to programmable devices 1004a-1004c. The content can include, but is not limited to, web pages, hypertext, scripts, binary data such as compiled software, images, audio, and/or video. ASCSS/ASK system 1010 can include one or more computing devices configured to perform some or all of the features described herein as being performed by an ASCSS, an ASK system, and/or a kiosk.

Although FIG. 10 only shows three client devices 1004a, 1004b, 1004c, distributed application architectures may serve tens, hundreds, or thousands of client devices. Moreover, client devices 1004a, 1004b, 1004c (or any additional client devices) may be any sort of computing device, such as an ordinary laptop computer, desktop computer, network terminal, wireless communication device (e.g., a cell phone or smart phone), and so on. In some embodiments, client devices 1004a, 1004b, 1004c can be dedicated to interacting with ASCSS/ASK system 1010. In other embodiments, client devices 1004a, 1004b, 1004c can be used as general purpose computers that are configured to perform a number of tasks and need not be dedicated to problem solving. In still other embodiments, part or all of the functionality of ASCSS/ASK system 1010 can be incorporated in a client device, such as client device 1004a, 1004b, and/or 1004c.

### Computing Device Architecture

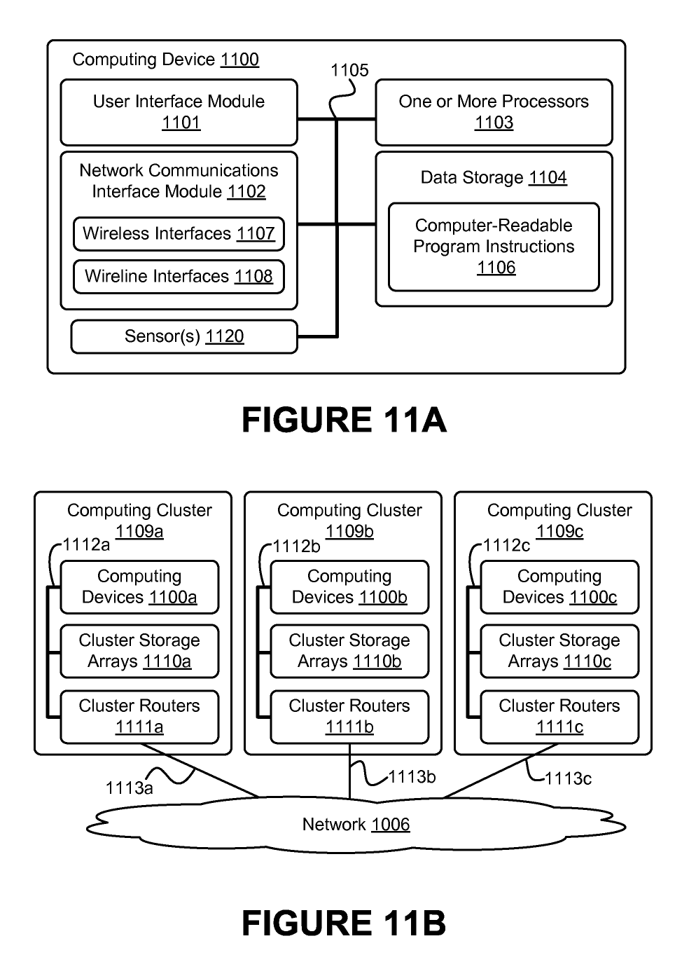

FIG. 11A is a block diagram of an example computing device (e.g., system). In particular, computing device 1100 shown in FIG. 11A can be configured to include components of and/or perform one or more functions or operations of kiosks 900, 960, 962, 964, 966, 968, operator interface 982, client device 1004a, 1004b, 1004c, network 1006, server 1008, and/or ASCSS/ASK system 1010 and/or carry out part or all of any herein-described studies and/or methods, such as but not limited to the first study, the second study, the third study and/or method 1200.

Computing device 1100 may include a user interface module 1101, a network-communication interface module 1102, one or more processors 1103, and data storage 1104, all of which may be linked together via a system bus, network, or other connection mechanism 1105. User interface module 1101 can be operable to send data to and/or receive data from external user input/output devices. For example, user interface module 1101 can be configured to send and/or receive data to and/or from user input devices such as a keyboard, a keypad, a touch screen, a computer mouse, a track ball, a joystick, a camera, a voice recognition module, and/or other similar devices. User interface module 1101 can also be configured to provide output to user display devices, such as one or more cathode ray tubes (CRT), liquid crystal displays (LCD), light emitting diodes (LEDs), displays using digital light processing (DLP) technology, printers, light bulbs, and/or other similar devices, either now known or later developed. User interface module 1101 can also be configured to generate audible output(s), such as a speaker, speaker jack, audio output port, audio output device, earphones, and/or other similar devices.

Network-communications interface module 1102 can include one or more wireless interfaces 1107 and/or one or more wireline interfaces 1108 that are configurable to communicate via a network, such as network 1006 shown in FIG. 10. Wireless interfaces 1107 can include one or more wireless transmitters, receivers, and/or transceivers, such as a Bluetooth transceiver, a Zigbee transceiver, a Wi-Fi transceiver, a WiMAX transceiver, and/or other similar type of wireless transceiver configurable to communicate via a wireless network. Wireline interfaces 1108 can include one or more wireline transmitters, receivers, and/or transceivers, such as an Ethernet transceiver, a Universal Serial Bus (USB) transceiver, or similar transceiver configurable to communicate via a twisted pair, one or more wires, a coaxial cable, a fiber-optic link, or a similar physical connection to a wireline network.

In some embodiments, network communications interface module 1102 can be configured to provide reliable, secured, and/or authenticated communications. For each communication described herein, information for ensuring reliable communications (i.e., guaranteed message delivery) can be provided, perhaps as part of a message header and/or footer (e.g., packet/message sequencing information, encapsulation header(s) and/or footer(s), size/time information, and transmission verification information such as CRC and/or parity check values). Communications can be made secure (e.g., be encoded or encrypted) and/or decrypted/decoded using one or more cryptographic protocols and/or algorithms, such as, but not limited to, DES, AES, RSA, Diffie-Hellman, and/or DSA. Other cryptographic protocols and/or algorithms can be used as well or in addition to those listed herein to secure (and then decrypt/decode) communications.

Processors 1103 can include one or more general purpose processors and/or one or more special purpose processors (e.g., digital signal processors, application specific integrated circuits, etc.). Processors 1103 can be configured to execute computer-readable program instructions 1106 contained in data storage 1104 and/or other instructions as described herein. Data storage 1104 can include one or more computer-readable storage media that can be read and/or accessed by at least one of processors 1103. The one or more computer-readable storage media can include volatile and/or non-volatile storage components, such as optical, magnetic, organic or other memory or disc storage, which can be integrated in whole or in part with at least one of processors 1103. In some embodiments, data storage 1104 can be implemented using a single physical device (e.g., one optical, magnetic, organic or other memory or disc storage unit), while in other embodiments, data storage 1104 can be implemented using two or more physical devices.

Data storage 1104 can include computer-readable program instructions 1106 and perhaps additional data. For example, in some embodiments, data storage 1104 can store part or all of data utilized by an ASCSS; e.g., ASCSS/ASK system 1010. In some embodiments, data storage 1104 can additionally include storage required to perform at least part of the herein-described methods and techniques and/or at least part of the functionality of the herein-described devices and networks.

In some embodiments, computing device 1100 can include one or more sensors 1120. Sensor(s) 1120 can be configured to measure conditions in an environment for computing device 1100 and/or persons in the environment and provide data about those person(s) in and/or the environment of computing device 1100. In some examples, sensor(s) 1120 can include one or more of: an eye-tracking device, a proximity sensor, a vibrometer, an eye-tracking sensor, a camera, an infrared sensor, an optical sensor, a light sensor, a biosensor, a capacitive sensor, a touch sensor, a temperature sensor, a wireless sensor, a radio sensor, a sound sensor, and/or a smoke sensor, possibly to obtain data indicative of an person(s) in and/or the environment of the computing device 1100. In particular examples, sensor(s) 1120 can determine credentials of person(s) in the environment about computing device 1100, including but not limited to one or more of: a card reader, a passport reader, biometric sensors (finger print readers, iris/eye blood vessel cameras/detectors, voice print detectors/analyzers), authentication chip readers e.g., radio-frequency identification (RFID) chip readers, keypads, cameras, and other sensor(s) configured to detect, process, and/or determine credentials of person(s) in the environment about computing device 1100. In other examples, sensor(s) 1120 can include one or more of: a gyroscope, an accelerometer, a Doppler sensor, a sonar sensor, a radar device, a laser-displacement sensor, and a compass, possibly to measure locations and/or movements of the computing device 900. Other examples of sensor(s) 1120 are possible as well.

For example, ASCSS/ASK system 1010 can be implemented using one or more computing devices, such as one or more computing devices 1100. The computing device(s) can be partially or wholly utilized as mobile computing device(s) (e.g., mobile devices, smart phones, tablets), portable computing device(s) (e.g., laptop computers), and/or other computing devices (e.g., embedded in one or more kiosks, desktop computers, mainframes). Other utilizations (form factors) for the computing device(s) is/are possible as well. The computing device(s) can include, communicate with, and/or otherwise exchange information from a number of sensors to assess credibility and/or detect the presence of concealed information. These sensor(s) can include, but are not limited to, one or more of sensors 1120 described herein.

FIG. 11B depicts a network 1006 of computing clusters 1109a, 1109b, 1109c arranged as a cloud-based server system in accordance with an example embodiment. Data and/or software for ASCSS/ASK system 1010 can be stored on one or more cloud-based devices that store program logic and/or data of cloud-based applications and/or services. In some embodiments, ASCSS/ASK system 1010 can be a single computing device residing in a single computing center. In other embodiments, ASCSS/ASK system 1010 can include multiple computing devices in a single computing center, or even multiple computing devices located in multiple computing centers located in diverse geographic locations.

In some embodiments, data and/or software for ASCSS/ASK system 1010 can be encoded as computer readable information stored in tangible computer readable media (or computer readable storage media) and accessible by client devices 1004a, 1004b, and 1004c, and/or other computing devices. In some embodiments, data and/or software for ASCSS/ASK system 1010 can be stored on a single disk drive or other tangible storage media, or can be implemented on multiple disk drives or other tangible storage media located at one or more diverse geographic locations.

FIG. 11B depicts a cloud-based server system in accordance with an example embodiment. In FIG. 11B, the operations of ASCSS/ASK system 1010 can be distributed among three computing clusters 1109a, 1109b, and 1109c. Computing cluster 1109a can include one or more computing devices 1100a, cluster storage arrays 1110a, and cluster routers 1111a connected by a local cluster network 1112a. Similarly, computing cluster 1109b can include one or more computing devices 1100b, cluster storage arrays 1110b, and cluster routers 1111b connected by a local cluster network 1112b. Likewise, computing cluster 1109c can include one or more computing devices 1100c, cluster storage arrays 1110c, and cluster routers 1111c connected by a local cluster network 1112c.

In some embodiments, each of the computing clusters 1109a, 1109b, and 1109c can have an equal number of computing devices, an equal number of cluster storage arrays, and an equal number of cluster routers. In other embodiments, however, each computing cluster can have different numbers of computing devices, different numbers of cluster storage arrays, and different numbers of cluster routers. The number of computing devices, cluster storage arrays, and cluster routers in each computing cluster can depend on the computing task or tasks assigned to each computing cluster.

In computing cluster 1109a, for example, computing devices 1100a can be configured to perform various computing tasks of ASCSS/ASK system 1010. In one embodiment, the various functionalities of ASCSS/ASK system 1010 can be distributed among one or more of computing devices 1100a, 1100b, and 1100c. Computing devices 1100b and 1100c in computing clusters 1109b and 1109c can be configured similarly to computing devices 1100a in computing cluster 1109a. On the other hand, in some embodiments, computing devices 1100a, 1100b, and 1100c can be configured to perform different operations.

In some embodiments, computing tasks and stored data associated with ASCSS/ASK system 1010 can be distributed across computing devices 1100a, 1100b, and 1100c based at least in part on the processing guidelines of ASCSS/ASK system 1010, the processing capabilities of computing devices 1100a, 1100b, and 1100c, the latency of the network links between the computing devices in each computing cluster and between the computing clusters themselves, and/or other factors that can contribute to the cost, speed, fault-tolerance, resiliency, efficiency, and/or other design goals of the overall system architecture.

The cluster storage arrays 1110a, 1110b, and 1110c of the computing clusters 1109a, 1109b, and 1109c can be data storage arrays that include disk array controllers configured to manage read and write access to groups of hard disk drives. The disk array controllers, alone or in conjunction with their respective computing devices, can also be configured to manage backup or redundant copies of the data stored in the cluster storage arrays to protect against disk drive or other cluster storage array failures and/or network failures that prevent one or more computing devices from accessing one or more cluster storage arrays.

Similar to the manner in which the operations of ASCSS/ASK system 1010 can be distributed across computing devices 1100a, 1100b, and 1100c of computing clusters 1109a, 1109b, and 1109c, various active portions and/or backup portions of these components can be distributed across cluster storage arrays 1110a, 1110b, and 1110c. For example, some cluster storage arrays can be configured to store one portion of the data and/or software of ASCSS/ASK system 1010, while other cluster storage arrays can store a separate portion of the data and/or software of ASCSS/ASK system 1010. Additionally, some cluster storage arrays can be configured to store backup versions of data stored in other cluster storage arrays.

The cluster routers 1111a, 1111b, and 1111c in computing clusters 1109a, 1109b, and 1109c can include networking equipment configured to provide internal and external communications for the computing clusters. For example, the cluster routers 1111a in computing cluster 1109a can include one or more internet switching and routing devices configured to provide (i) local area network communications between the computing devices 1100a and the cluster storage arrays 1110a via the local cluster network 1112a, and (ii) wide area network communications between the computing cluster 1109a and the computing clusters 1109b and 1109c via the wide area network connection 1113a to network 1006. Cluster routers 1111b and 1111c can include network equipment similar to the cluster routers 1111a, and cluster routers 1111b and 1111c can perform similar networking operations for computing clusters 1109b and 1109b that cluster routers 1111a perform for computing cluster 1109a.

In some embodiments, the configuration of the cluster routers 1111a, 1111b, and 1111c can be based at least in part on the data communication requirements of the computing devices and cluster storage arrays, the data communications capabilities of the network equipment in the cluster routers 1111a, 1111b, and 1111c, the latency and throughput of local networks 1112a, 1112b, 1112c, the latency, throughput, and cost of wide area network links 1113a, 1113b, and 1113c, and/or other factors that can contribute to the cost, speed, fault-tolerance, resiliency, efficiency and/or other design goals of the moderation system architecture.

### Example Methods of Operation

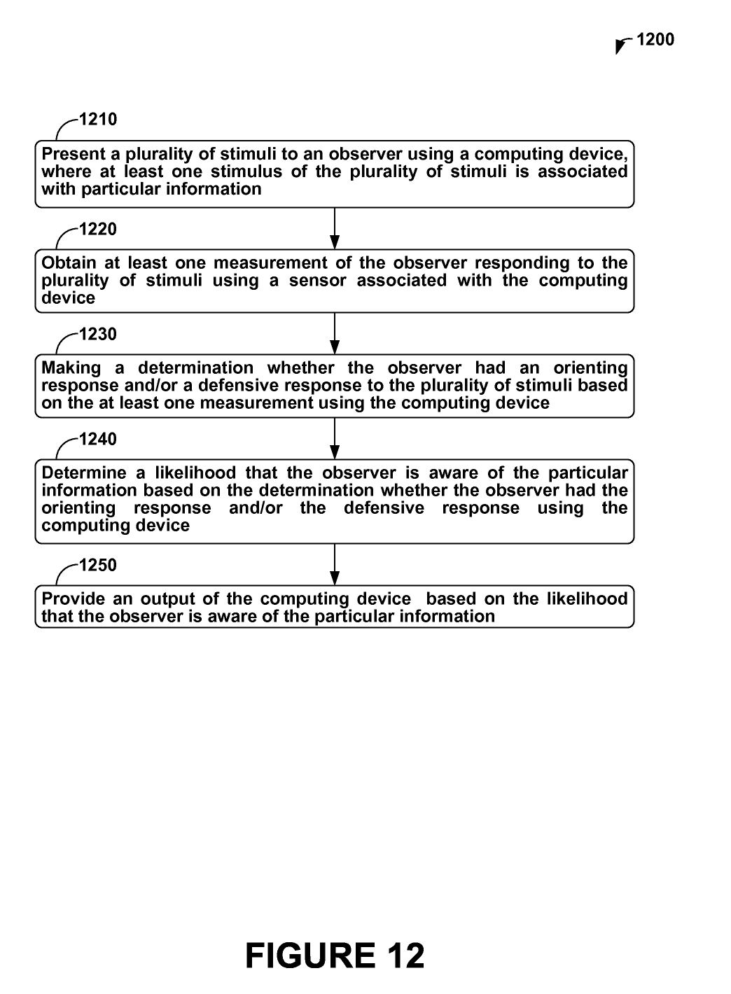

FIG. 12 is a flow chart of an example method 1200. Method 1200 can be carried out by a computing device, such as a computing device 1100. The computing device can be configured with at least some of the herein-described functionality, including but not limited to, functionality related to ASCSS and/or an ASK system. In some embodiments, the computing device can be configured to be operated in a kiosk; e.g., kiosk 900 discussed above in the context of at least FIG. 9A.

Method 1200 can begin at block 1210, where the computing device can present a plurality of stimuli to an observer (such as but not limited to a human examinee), where at least one stimulus of the plurality of stimuli can be associated with particular information. In some embodiments, the plurality of stimuli can include visual stimuli. In particular of these embodiments, the plurality of stimuli can include a plurality of images.

At block 1220, the computing device can obtain at least one measurement of the observer responding to the plurality of stimuli using a sensor associated with the computing device. In some embodiments, the at least one measurement can include a measurement of eye movement of the observer.

At block 1230, the computing device can make a determination whether the observer had an orienting response and/or a defensive response to the plurality of stimuli based on the at least one measurement.

In some embodiments, making the determination whether the observer had the orienting response and/or the defensive response to the plurality of stimuli can include determining whether the observer had the orienting response based on an initial response to the plurality of stimuli. In particular of these embodiments, the plurality of stimuli can include a plurality of images, where a single particular image of the plurality of images can be associated with the particular information. Then, determining whether the observer had the orienting response based on an initial response to the plurality of stimuli can include determining whether an initial saccade of the observer is directed to the single particular image.

In other embodiments, making the determination whether the observer had the orienting response and/or the defensive response to the plurality of stimuli can include determining whether the observer had the defensive response based on a response to the plurality of stimuli. In particular of these embodiments, the plurality of stimuli can include a plurality of images, where a single particular image of the plurality of images is associated with the particular information. Then, determining whether the observer had the defensive response can include determining whether the observer made at least one eye movement to a location away from the single particular image. In more particular of these embodiments, presenting the plurality of stimuli includes presenting the plurality of images in locations with respect to a location of a fixation marker, and where the location away from the single particular image includes the location of the fixation marker.

At block 1240, the computing device can determine a likelihood that the observer is aware of the particular information based on the determination whether the observer had the orienting response and/or the defensive response.

At block 1250, the computing device can provide an output based on the likelihood that the observer is aware of the particular information.

In some embodiments, providing the output of the computing device based on the likelihood that the observer is aware of the particular information can include: determining whether the likelihood that the observer is aware of the particular information is above a threshold likelihood; and after determining that likelihood that the observer is aware of the particular information is above the threshold likelihood, providing an output of the computing device recommending further screening of the observer.

In other embodiments, providing the output of the computing device based on the likelihood that the observer is aware of the particular information can include: determining that the likelihood that the observer is aware of the particular information is not above a threshold likelihood; and after determining that the likelihood that the observer is aware of the particular information is not above the threshold likelihood, providing an output of the computing device indicating the observer likely does not have the particular information and so recommending no additional screening of the observer.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The above description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure. The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The above detailed description describes various features and functions of the disclosed systems, devices, and methods with reference to the accompanying figures. In the figures, similar symbols typically identify similar components, unless context dictates otherwise. The illustrative embodiments described in the detailed description, figures, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the ladder diagrams, scenarios, and flow charts in the figures and as discussed herein, each block and/or communication may represent a processing of information and/or a transmission of information in accordance with example embodiments. Alternative embodiments are included within the scope of these example embodiments. In these alternative embodiments, for example, functions described as blocks, transmissions, communications, requests, responses, and/or messages may be executed out of order from that shown or discussed, including substantially concurrent or in reverse order, depending on the functionality involved. Further, more or fewer blocks and/or functions may be used with any of the ladder diagrams, scenarios, and flow charts discussed herein, and these ladder diagrams, scenarios, and flow charts may be combined with one another, in part or in whole.

A block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical functions or actions in the method or technique. The program code and/or related data may be stored on any type of computer readable medium such as a storage device including a disk or hard drive or other storage medium.

The computer readable medium may also include non-transitory computer readable media such as computer-readable media that stores data for short periods of time like register memory, processor cache, and random access memory (RAM). The computer readable media may also include non-transitory computer readable media that stores program code and/or data for longer periods of time, such as secondary or persistent long term storage, like read only memory (ROM), optical or magnetic disks, compact-disc read only memory (CD-ROM), for example. The computer readable media may also be any other volatile or non-volatile storage systems. A computer readable medium may be considered a computer readable storage medium, for example, or a tangible storage device.

Moreover, a block that represents one or more information transmissions may correspond to information transmissions between software and/or hardware modules in the same physical device. However, other information transmissions may be between software modules and/or hardware modules in different physical devices.

Numerous modifications and variations of the present disclosure are possible in light of the above teachings.

1. A method, comprising:
presenting a plurality of stimuli to an observer using a computing device, wherein at least one stimulus of the plurality of stimuli is associated with particular information;
obtaining at least one measurement of the observer responding to the plurality of stimuli using a sensor associated with the computing device;
making a determination whether the observer had an orienting response and/or a defensive response to the plurality of stimuli based on the at least one measurement using the computing device;
determining a likelihood that the observer is aware of the particular information based on the determination whether the observer had the orienting response and/or the defensive response using the computing device; and
providing an output of the computing device based on the likelihood that the observer is aware of the particular information.

2. The method of claim 1, wherein the plurality of stimuli comprise visual stimuli.

3. The method of claim 1, wherein the plurality of stimuli comprise a plurality of images.

4. The method of claim 1, wherein the at least one measurement comprises a measurement of eye movement of the observer.

5. The method of claim 1, wherein making the determination whether the observer had the orienting response and/or the defensive response to the plurality of stimuli comprises determining whether the observer had the orienting response based on an initial response to the plurality of stimuli.

6. The method of claim 5, wherein the plurality of stimuli comprise a plurality of images, wherein a single particular image of the plurality of images is associated with the particular information, and wherein determining whether the observer had the orienting response based on an initial response to the plurality of stimuli comprises determining whether an initial saccade of the observer is directed to the single particular image.

7. The method of claim 1, wherein determining whether the observer had the orienting response and/or the defensive response to the plurality of stimuli comprises:
determining whether the observer had the defensive response based on a response to the plurality of stimuli.

8. The method of claim 7, wherein the plurality of stimuli comprise a plurality of images, wherein a single particular image of the plurality of images is associated with the particular information, and wherein determining whether the observer had the defensive response comprises determining whether the observer made at least one eye movement to a location away from the single particular image.

9. The method of claim 8, wherein presenting the plurality of stimuli comprises presenting the plurality of images in locations with respect to a location of a fixation marker, and wherein the location away from the single particular image comprises the location of the fixation marker.

10. The method of claim 1, wherein providing the output of the computing device based on the likelihood that the observer is aware of the particular information comprises:
determining whether the likelihood that the observer is aware of the particular information is above a threshold likelihood; and
after determining that likelihood that the observer is aware of the particular information is above the threshold likelihood, providing an output of the computing device recommending further screening of the observer.

11. The method of claim 1, wherein providing the output of the computing device based on the likelihood that the observer is aware of the particular information comprises:
determining that the likelihood that the observer is aware of the particular information is not above a threshold likelihood; and
after determining that the likelihood that the observer is aware of the particular information is not above the threshold likelihood, providing an output of the computing device indicating the observer likely does not have the particular information and so recommending no additional screening of the observer.

12. A computing device, comprising:
a sensor;
a processor; and
a non-transitory computer-readable medium configured to store instructions that, when executed by the processor, are configured to cause the computing device to perform functions, comprising:
presenting a plurality of stimuli to an observer, wherein at least one stimulus of the plurality of stimuli is associated with particular information;
obtaining at least one measurement of the observer responding to the plurality of stimuli using the sensor;
making a determination whether the observer had an orienting response and/or a defensive response to the plurality of stimuli based on the at least one measurement;
determining a likelihood that the observer is aware of the particular information based on the determination whether the observer had the orienting response and/or the defensive response; and
providing an output that is based on the likelihood that the observer is aware of the particular information.

13. (canceled)

14. The computing device of claim 12, wherein the computing device is configured to be operated in a kiosk.

15-18. (canceled)

19. The computing device of claim 12, wherein the plurality of stimuli comprise a plurality of images.

20. The computing device of claim 12, wherein the at least one measurement comprises a measurement of eye movement of the observer.

21. The computing device of claim 12, wherein making the determination whether the observer had the orienting response and/or the defensive response to the plurality of stimuli comprises determining whether the observer had the orienting response based on an initial response to the plurality of stimuli.

22. The computing device of claim 12, wherein determining whether the observer had the orienting response and/or the defensive response to the plurality of stimuli comprises:
determining whether the observer had the defensive response based on a response to the plurality of stimuli.

23. The computing device of claim 12, wherein providing the output of the computing device based on the likelihood that the observer is aware of the particular information comprises:
determining whether the likelihood that the observer is aware of the particular information is above a threshold likelihood; and
after determining that likelihood that the observer is aware of the particular information is above the threshold likelihood, providing an output of the computing device recommending further screening of the observer.

24. The computing device of claim 12, wherein providing the output of the computing device based on the likelihood that the observer is aware of the particular information comprises:
determining that the likelihood that the observer is aware of the particular information is not above a threshold likelihood; and
after determining that the likelihood that the observer is aware of the particular information is not above the threshold likelihood, providing an output of the computing device indicating the observer likely does not have the particular information and so recommending no additional screening of the observer.

25. A non-transitory computer-readable medium configured to store instructions that, when executed by a processor of a computing device, are configured to cause the computing device to perform functions, comprising:
presenting a plurality of stimuli to an observer, wherein at least one stimulus of the plurality of stimuli is associated with particular information;
obtaining at least one measurement of the observer responding to the plurality of stimuli using the sensor;
making a determination whether the observer had an orienting response and/or a defensive response to the plurality of stimuli based on the at least one measurement;
determining a likelihood that the observer is aware of the particular information based on the determination whether the observer had the orienting response and/or the defensive response; and
providing an output that is based on the likelihood that the observer is aware of the particular information.
